(19) United States
(12) Patent Application Publication
(10) Pub. No.: US 2023/0213532 A1
(43) Pub. Date: Jul. 6, 2023

(54) ASSESSMENT METHODS AND DIAGNOSTIC KIT FOR PREDICTING SUICIDAL BEHAVIORS IN PATIENTS WITH DEPRESSIVE DISORDERS USING MULTIMODAL SERUM BIOMARKERS

(71) Applicants: INDUSTRY FOUNDATION OF CHONNAM NATIONAL UNIVERSITY, Gwangju (KR); CHONNAM NATIONAL UNIVERSITY HOSPITAL, Gwangju (KR)

(72) Inventors: Jae Min KIM, Gwangju (KR); Hee Ju KANG, Gwangju (KR)

(21) Appl. No.: 17/710,184

(22) Filed: Mar. 31, 2022

(30) Foreign Application Priority Data
Jan. 5, 2022 (KR) 10-2022-0001366

(51) Int. Cl. G01N 33/68; G01N 33/74; G01N 33/92; G01N 33/82

(52) U.S. Cl. CPC G01N 33/6893; G01N 33/6869; G01N 33/743; G01N 33/92; G01N 33/82; G01N 2800/304; G01N 2333/545

(57) ABSTRACT

A method for assessing a suicidal severity in a depressed patient according to an embodiment includes measuring a concentration of a suicidal behavior prediction biomarker contained in a biological sample of the depressed patient, wherein the suicidal behavior prediction biomarker is one or more markers selected from the group consisting of cortisol, interleukin-1 beta (IL-1β), homocysteine, total cholesterol, and folate, and determining a probability of an increased suicidal severity by comparing the measured concentration of the suicidal behavior prediction biomarker with a preset cutoff level thereof.

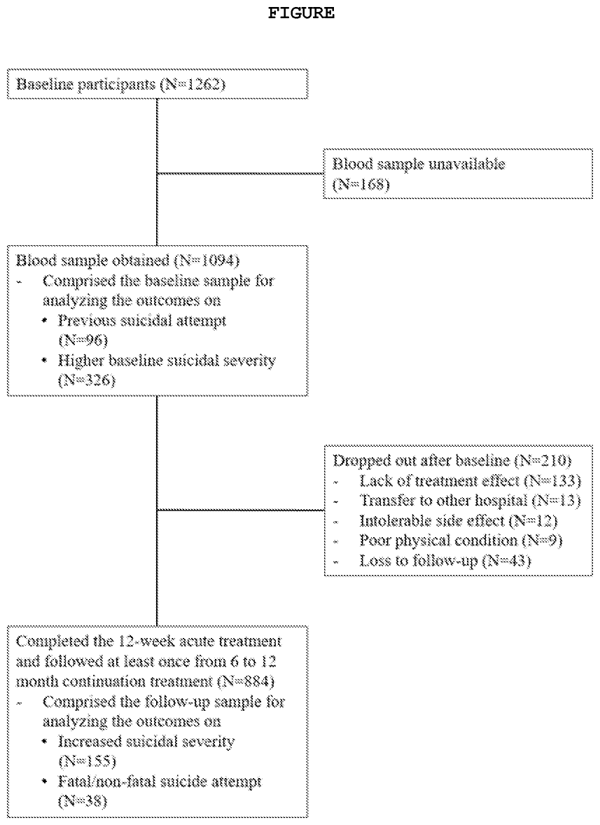

### CROSS REFERENCE TO RELATED APPLICATION

This application claims the benefit under 35 USC § 119 of Korean Patent Application No. 10-2022-0001366, filed on Jan. 5, 2022, at the Korean Intellectual Property Office, the entire disclosure of which is incorporated herein by reference for all purposes.

### BACKGROUND

### 1. Field of the Invention

The present invention relates to a diagnostic method for predicting suicidal behaviors in patients with depressive disorders, and more particularly to assessment methods for predicting suicidal behaviors(including an increased suicidal severity and a fatal/non-fatal suicide attempt: SB) of depressed patients receiving drug therapy by examining biomarkers contained in biological samples of depressed patients at baseline, and a diagnostic kit.

### 2. Description of the Related Art

Suicide is a major cause of death globally in that approximately 800,000 people die by suicide every year (Naghavi et al. 2019). Suicidal ideation and attempt are 10-20 times more common than fatal suicide (Mann 2003). A rational first step for monitoring and preventing suicidal behaviour (SB) is the identification of risk factors, which is not easy because it relies on subjective reports (Blasco-Fontecilla et al. 2013). Since suicide has distinctive pathophysiologies based on stress-diathesis models (Oquendo et al. 2014), application of objective biological tests could improve the predictability of SB (Sudol & Mann 2017).

Among the biological measures, peripheral blood biomarkers have several advantages, given their accessibility, cost-effectiveness, and ease of collection even in those with suicidal risks. A variety of peripheral blood biomarkers relevant to pathophysiologies of SB was evaluated. Markers related to the hypothalamic-pituitary-adrenal(HPA) axis, a major stress-response system, has been researched extensively. Cortisol is an effector hormone of HPA axis, but its relations with SB have been inconsistent (O'Connor et al. 2016). Serotonergic system is involved in both stress and diathesis of SB (Mann 2013), and low blood serotonin levels were related to SB (Tyano et al. 2006). Markers of immune and inflammatory function have long been investigated, given their connection to the HPA axis and serotonin system. The main markers studied were high-sensitivity C-reactive protein (hsCRP), pro-inflammatory cytokines including tumor necrosis factor-alpha (TNF-α), interleukin-1 beta (IL-1β), IL-6, etc., and anti-inflammatory cytokines including IL-4, IL-10, etc. (Black & Miller 2014; Choi et al. 2021). Lipids may also play a role in the pathophysiology of SB, given that low cholesterol levels could impair serotonin transportation in the central nervous system (CNS) (Engelberg 1992; Wu et al. 2016). Leptin and ghrelin, which could affect lipid concentrations have also been investigated (Gonzalez-Castro et al. 2020; Atmaca et al. 2006). Nutrients that could protect against cellular damage by stresses and affective disorders could be biomarker candidates including folate, omega 3 fatty acid, homocysteine, etc. (Du et al. 2016). Neuroplastic function is important in adaptation of CNS to external stresses, and brain-derived neurotrophic factor (BDNF) was the most frequently studied (Eisen et al. 2015).

Despite of extensive previous researches, the blood biomarkers suggested are rarely used in clinical practice following reasons. First, diagnostic and screening values of the individual biomarkers were disappointing to gauge risks of SB (Blasco-Fontecilla & Oquendo 2017). Combination of two unrelated biomarkers might provide better results than a single one, but the accuracy was still unsatisfactory (Coryell & Schlesser 2007; Jokinen et al. 2008). An investigation of multiple biomarkers covering various functional systems in combination might increase the accuracy (Sudol & Mann 2017), while this approach has not been conducted. Second, most studies evaluated the SB as previous suicidal attempt or present suicidal severity with case-control study designs rather than as prospective suicidality (O'Connor et al. 2016; Black & Miller 2014; Wu et al. 2016; Eisen et al. 2015). The findings from these approaches might detect past or present SB but could not predict future SB accurately.

### SUMMARY

The present inventors have performed research to predict suicidal behavior of depressed patients based on the concentration of specific biomarkers present in biological samples of depressed patients, thus culminating in the present invention.

Accordingly, an aspect of the present invention is to provide an assessment method for predicting suicidal behavior of depressed patients at a baseline by specifying one or more biomarkers and cut-off levels for predicting suicidal behavior in depressed patients, which may contribute to a decision-making process with regard to therapeutic drugs or treatment methods. This is because the possibility of suicidal behavior occurring during drug therapy was confirmed through an experiment when a specific biomarker was present at a specific concentration, that is, below or exceeding the cut-off level, in the biological sample of depressed patients at the baseline.

Another aspect of the present invention is to provide a diagnostic kit for predicting the suicidal behavior of depressed patients by measuring a concentration of one or more suicidal behavior prediction biomarkers contained in a biological sample of a depressed patient at a baseline, in which the suicidal behavior prediction biomarkers include cortisol, interleukin-1 beta (IL-1β), homocysteine, total cholesterol, and folate, and providing a customized treatment strategy to each patient based on the prediction result, thus providing clinical usefulness.

The aspects of the present invention are not limited to the foregoing, and it is to be understood that other aspects not mentioned herein can be clearly anticipated by those skilled in the art from the following description.

In order to accomplish one or more of the above aspects, the present invention provides an assessment method for predicting suicidal behavior in depressed patients, including a measurement step of measuring a concentration of a suicidal behavior prediction biomarker contained in a biological sample of a depressed patient at a baseline, and a decision step of determining a probability of an increased suicidal severity, depending on the concentration of the suicidal behavior prediction biomarker.

In an exemplary embodiment, the suicidal behavior prediction biomarker is one or more markers selected from the group consisting of cortisol, interleukin-1 beta (IL-1β), homocysteine, total cholesterol, and folate.

In an exemplary embodiment, the decision step is performed by comparing the measured concentration of the suicidal behavior prediction biomarker with a preset cutoff level thereof, wherein when the measured concentration of each of the cortisol, interleukin-1 beta (IL-1β), and homocysteine is higher than the preset cutoff level thereof, it is determined that an increased suicidal severity is likely to occur, and when the concentration of each of the total cholesterol and folate is lower than the preset cutoff level, it is determined that an increased suicidal severity is likely to occur.

In an exemplary embodiment, when the suicidal behavior prediction biomarker is the cortisol, the preset cutoff level is 11.7 μg/dL, when the suicidal behavior prediction biomarker is the IL-1β, the preset cutoff level is 0.99 μg/mL, when the suicidal behavior prediction biomarker is the homocysteine, the preset cutoff level is 11.1 μmol/dL, when the suicidal behavior prediction biomarker is the total cholesterol, the preset cutoff level is 155.0 mg/dL, and when the suicidal behavior prediction biomarker is the folate, the preset cutoff level is 6.05 ng/mL.

In an exemplary embodiment, in the decision step, as the number of the suicidal behavior prediction biomarkers indicating that an increased suicidal severity is likely to occur increases, the probability of an increased suicidal severity increases compared to a reference level.

In an exemplary embodiment, when the number of the suicidal behavior prediction biomarkers indicating that an increased suicidal severity is likely to occur is one, the probability of an increased suicidal severity increases 2.1 times compared to the reference level, and when the number of the suicidal behavior prediction biomarkers is five, the probability of an increased suicidal severity increases 16.1 times compared to the reference level.

In addition, the present invention provides an assessment method for predicting suicidal behavior in depressed patients, including a measurement step of measuring a concentration of each of five suicidal behavior prediction biomarkers contained in a biological sample of a depressed patient at a baseline, the five suicidal behavior prediction biomarkers including cortisol, interleukin-1 beta (IL-1β), homocysteine, total cholesterol, and folate, and a decision step of determining a probability of an increased suicidal severity, depending on the concentration of each of the suicidal behavior prediction biomarkers.

In an exemplary embodiment, the decision step includes a reference point allocation step, a calculation step and a determination step. In the reference point allocation step, for each of the respective measured concentrations of the cortisol, IL-1β, and homocysteine, a reference point of 1 is allocated when the measured concentration is higher than the preset cutoff level thereof and a reference point of 0 is allocated when the measured concentration is lower than the preset cutoff level, and for each of the respective measured concentrations of the total cholesterol and folate, a reference point of 1 is allocated when the measured concentration is lower than the preset cutoff level and a reference point of 0 is allocated when the measured concentration is higher than the preset cutoff level. The calculation step calculates a multi-biomarker score according to Formula 1 below.

multi-biomarker score=1.108×A+0.700×B+0.331×C+0.193×D+0.282×E, [Formula 1]

wherein A is the reference point of the IL-1β, B is the reference point of the total cholesterol, C is the reference point of the cortisol, D is the reference point of the folate, and E is the reference point of the homocysteine. In the determination step, a probability of an increased suicidal severity is determined by finding a quartile in which the calculated total score is located.

In an exemplary embodiment, when the suicidal behavior prediction biomarker is the cortisol, the preset cutoff level is 11.7 μg/dL, when the suicidal behavior prediction biomarker is the IL-1β, the preset cutoff level is 0.99 μg/mL, when the suicidal behavior prediction biomarker is the homocysteine, the preset cutoff level is 11.1 μmol/dL, when the suicidal behavior prediction biomarker is the total cholesterol, the preset cutoff level is 155.0 mg/dL, and when the suicidal behavior prediction biomarker is the folate, the preset cutoff level is 6.05 ng/mL.

In an exemplary embodiment, when the multi-biomarker score is within a range of 0 to 0.61, the score is located in the first quartile, when the multi-biomarker score is within a range of 0.62 to 1.31, the score is located in the second quartile, when the multi-biomarker score is within a range of 1.32 to 1.72, the score is located in the third quartile, and when the multi-biomarker score is within a range of 1.73 to 2.61, the score is located in the fourth quartile.

In an exemplary embodiment, wherein when the score is located in the first quartile, the probability of an increased suicidal severity is less than 6%.

In an exemplary embodiment, when the score is located in the second quartile, the probability of an increased suicidal severity increases 2.20 times compared to the first quartile, when the score is located in the third quartile, the probability of an increased suicidal severity increases 4.29 times compared to the first quartile, and when the score is located in the fourth quartile, the probability of an increased suicidal severity increases 6.20 times compared to the first quartile.

In addition, the present invention provides an assessment method for predicting suicidal behavior in depressed patients, including a measurement step of measuring the concentration of a suicidal behavior prediction biomarker contained in a biological sample of a depressed patient at a baseline, and a decision step of determining a probability of occurrence of a fatal/non-fatal suicide attempt according to the concentration of the suicidal behavior prediction biomarker.

In an exemplary embodiment, the suicidal behavior prediction biomarker is one or more markers selected from the group consisting of cortisol, total cholesterol, and folate.

In an exemplary embodiment, the decision step is performed by comparing the measured concentration of the suicidal behavior prediction biomarker with a preset cutoff level thereof, wherein when the concentration of the cortisol is higher than the preset cutoff level, it is determined that a fatal/non-fatal suicide attempt is likely to occur, and when the concentration of each of the total cholesterol and folate is lower than the preset cutoff level thereof, it is determined that a fatal/non-fatal suicide attempt is likely to occur.

In an exemplary embodiment, when the suicidal behavior prediction biomarker is the cortisol, the preset cutoff level is 12.0 μg/dL, when the suicidal behavior prediction biomarker is the total cholesterol, the preset cutoff level is 154.0 mg/dL, and when the suicidal behavior prediction biomarker is the folate, the preset cutoff level is 5.95 ng/mL.

In an exemplary embodiment, in the decision step, as the number of suicidal behavior prediction biomarkers indicating that a fatal/non-fatal suicide attempt is likely to occur increases, the probability of occurrence of a fatal/non-fatal suicide attempt increases compared to a reference level.

In an exemplary embodiment, when the number of suicidal behavior prediction biomarkers indicating that a fatal/non-fatal suicide attempt is likely to occur is one, the probability of a fatal/non-fatal suicide attempt increases 3.3 times compared to the reference level, and when the number of the suicidal behavior prediction biomarkers is three, the probability of a fatal/non-fatal suicide attempt increases 63.3 times compared to the reference level.

In addition, the present invention provides an assessment method for predicting suicidal behavior in depressed patients, including a measurement step of measuring the concentration of each of three suicidal behavior prediction biomarkers contained in a biological sample of a depressed patient at a baseline, the three suicidal behavior prediction biomarkers including cortisol, total cholesterol, and folate, and a decision step of determining a probability of a fatal/non-fatal suicide attempt according to the measured concentration of each of the suicidal behavior prediction biomarkers.

In an exemplary embodiment, the decision step includes a reference point allocation step, a calculation step and a determination step. In the reference point allocation step, for the measured concentration of the cortisol, a reference point of 1 is allocated when the measured concentration is higher than the preset cutoff level thereof and a reference point of 0 is allocated when the measured concentration is lower than the preset cutoff level thereof, and for each of the respective measured concentrations of the total cholesterol and folate, a reference point of 1 is allocated when the measured concentration is lower than the preset cutoff level thereof and a reference point of 0 is allocated when the measured concentration is higher than the preset cutoff level thereof. The calculation step calculates a multi-biomarker score according to Formula 2 below.

multi-biomarker score=1.215×B+1.843×C+1.010×D, [Formula 2]

wherein B is the reference point of the total cholesterol, C is the reference point of the cortisol, and D is the reference point of the folate. In the determination step, a probability of a fatal/non-fatal suicide attempt is determined by finding a quartile in which the calculated total score is located.

In an exemplary embodiment, when the suicidal behavior prediction biomarker is the cortisol, the preset cutoff level is 12.0 μg/dL, when the suicidal behavior prediction biomarker is the total cholesterol, the preset cutoff level is 154.0 mg/dL, and when the suicidal behavior prediction biomarker is the folate, the preset cutoff level is 5.95 ng/mL.

In an exemplary embodiment, when the multi-biomarker score is 0, the score is located in the first quartile, when the multi-biomarker score is within a range of 0.01 to 1.21, the score is located in the second quartile, when the multi-biomarker score is within a range of 1.22 to 1.84, the score is located in the third quartile, and when the multi-biomarker score is within a range of 1.85 to 4.07, the score is located in the fourth quartile.

In an exemplary embodiment, when the score is located in the first quartile, the probability of a fatal/non-fatal suicide attempt is less than 0.5%.

In an exemplary embodiment, when the score is located in the second quartile, the probability of a fatal/non-fatal suicide attempt increases 0.99 times compared to the first quartile, when the score is located in the third quartile, the probability of a fatal/non-fatal suicide attempt increases 5.36 times compared to the first quartile, and when the score is located in the fourth quartile, the probability of a fatal/non-fatal suicide attempt increases 32.34 times compared to the first quartile.

In addition, the present invention provides a diagnostic kit for predicting suicidal behavior in depressed patients, including a biomarker measurement unit configured to measure the concentration of one or more biomarkers contained in a biological sample of a depressed patient, the one or more biomarkers being selected from the group consisting of cortisol, interleukin-1 beta (IL-1β)), homocysteine, total cholesterol, and folate.

In an exemplary embodiment, the biomarker measurement unit measures the concentration of each of the biomarkers using a high-sensitivity bead panel utilizing, ELISA, ECLISA, or an enzymatic method.

In an exemplary embodiment, the biological sample of the depressed patient is serum.

In an exemplary embodiment, the diagnostic kit is a microarray.

In an exemplary embodiment, when the suicidal behavior is an increased suicidal severity, the suicidal behavior of the depressed patient is predicted according to the assessment method.

In an exemplary embodiment, when the suicidal behavior is a fatal/non-fatal suicide attempt, the suicidal behavior of the depressed patient is predicted according to the assessment method.

According to the present invention, it is confirmed that whether the concentration of one or more markers below or above a specific concentration selected from the group consisting of cortisol, interleukin-1 beta (IL-1β), homocysteine, total cholesterol, and folate present in biological samples of depressed patients at a baseline can be used as a biomarker for predicting suicidal behaviors(including an increased suicidal severity and a fatal/non-fatal suicide attempt) of depressed patients receiving drug therapy, thereby providing a biomarker that can relatively accurately determine a probability of suicidal behaviors in depressed patients at a baseline.

In addition, an assessment method according to the present invention enables the prediction and/or diagnosis of the possibility of predicting suicidal behavior of depressed patients at a baseline by specifying one or more biomarkers and cutoff levels for predicting suicidal behavior in depressed patients and can thus contribute to the decision-making process of the doctor with regard to therapeutic treatment strategy, considering patients with adverse biomarkers should be monitored frequently and treated carefully to prevent SB.

Moreover, a diagnostic kit according to the present invention enables the prediction and/or diagnosis of the possibility of predicting suicidal behavior of depressed patients by measuring a concentration of one or more suicidal behavior prediction biomarkers contained in a biological sample of a depressed patient at a baseline, and providing a customized treatment strategy to each patient based on the prediction result, thus providing clinical usefulness.

The effects of the present invention are not limited to the foregoing, and it is to be understood that other objects not mentioned herein can be clearly anticipated by those skilled in the art from the following description.

### BRIEF DESCRIPTION OF THE DRAWING

FIGURE shows a flow chart depicting each participant in the treatment phase over a 12-month drug therapy period.

### DETAILED DESCRIPTION

The terminology used in the present invention is merely used to describe particular embodiments, and is not intended to limit the present invention. As used herein, the singular forms are intended to include the plural forms as well, unless the context clearly indicates otherwise. It will be understood that the terms “comprise”, “include”, “have”, etc. when used in this specification specify the presence of stated features, integers, steps, operations, elements, components, or combinations thereof, but do not preclude the presence or addition of one or more other features, integers, steps, operations, elements, components, or combinations thereof.

It will be further understood that, although terms such as “first”, “second”, etc. may be used herein to describe various elements, these elements are not to be limited by these terms. These terms are only used to distinguish one element from another element. For instance, a “first” element discussed below could be termed a “second” element without departing from the scope of the present invention. Similarly, the “second” element could also be termed a “first” element.

Unless otherwise defined, all terms including technical and scientific terms used herein have the same meanings as those commonly understood by one of ordinary skill in the art to which the present invention belongs. It will be further understood that the terms used herein should be interpreted as having meanings consistent with their meanings in the context of this specification and the relevant art, and are not to be interpreted in an idealized or overly formal sense unless expressly so defined herein.

In interpreting elements, it is to be understood that an error range is included even if there is no separate description thereof.

In the case of a description of a temporal relationship, for example, when the temporal relationship is described as ‘after’, ‘following’, ‘subsequently, ‘before’, etc., this includes non-consecutive cases, unless ‘immediately or ‘directly’ is used.

As used herein, the term “diagnosis” means identifying the presence or characteristic of a pathological condition. With regard to the purpose of the present invention, “diagnosis” means determining a probability of suicidal behavior in depressed patients receiving drug therapy based on in vitro analysis of a biological sample of depressed patients at a baseline, in which the probability of suicidal behavior means the probability of suicidal behavior occurring within 12 months from the baseline.

As used herein, the term “biomarker” means a substance that may indicate a disease state. In the context of the present invention regarding the diagnosis of predicting suicidal behavior in depressed patients, the “biomarker” means that at least one selected from the group consisting of cortisol, interleukin-1 beta (IL-1β)), homocysteine, total cholesterol, and folate has a concentration below the preset cut-off level or a concentration above the preset cut-off level. Among depressed patients at a baseline, as the number of the biomarkers increases, the probability of suicidal behavior increases compared to patients with a reference level without any biomarkers.

As used herein, the term “cutoff level” or “the preset cutoff level” means the relative or absolute level determined to distinguish between individuals with the potential for suicidal behavior within 12 months from the baseline of depressed patients receiving drug therapy. The cutoff level can be given as a value expressed as a fold difference in the case of a relative level or as a concentration for example in the case of an absolute value. As pointed out herein, depending on the biomarker, values below or above the cut-off level are considered to determine the probability of suicidal behavior within 12 months at a baseline.

As used herein, the term “reference level” refers to a state in which there is no biomarker in a biological sample of a depressed patient at a baseline. That is, it means a state in which all of the above-mentioned five biomarker concentrations are above or below the cutoff level determined for each biomarker.

As used herein, the term “predicting” refers to discovering that an individual is more likely to develop suicidal behavior or is less likely to develop suicidal behavior during drug therapy.

As used herein, the term “biological sample” includes various types of samples obtained from an individual, and may also be used in diagnosis or monitoring analysis. Biological fluid samples include blood, cerebrospinal fluid (CSF), urine, and other liquid samples of biological origin. For example, the sample may be pretreated for concentration and separation, if necessary.

As used herein, the term “blood” includes whole blood, serum and plasma.

As used herein, the term “individual” is a mammal, preferably a human, and the terms “individual” and “subject” may be used interchangeably in the present invention.

As used herein, the term “baseline” refers to the time point at which initial medical treatment for drug therapy is performed for a depressed patient.

Hereinafter, a detailed description will be given of the technical configuration of the present invention with reference to the accompanying drawings and exemplary embodiments.

However, the present invention is not limited to the embodiments described herein, and may be embodied in other forms. Throughout the specification, the same reference numerals used to explain the present invention designate the same elements.

The present inventors have ascertained that the concentration of one or more of Cortisol, interleukin-1 beta (IL-1β), homocysteine, total cholesterol and folic acid below or above the cut-off level in biological samples from depressed patients at baseline may be used as a biomarker for predicting suicidal behavior in depressed patients. Thus, the present invention provides a method for predicting and/or diagnosing suicidal behavior in depressed patients at the baseline of starting drug therapy using, as a biomarker, the concentration below or above the cut-off level of one or more of Cortisol, interleukin-1 beta (IL-1β), homocysteine, total cholesterol and folic acid, and a diagnostic kit.

In the present invention, as described below, the effect of whether the concentration of one or more of cortisol, interleukin-1 beta (IL-1β), and homocysteine present in a biological sample of a depressed patient at a baseline is above a cut-off level and/or whether the concentration of one or more of total cholesterol and folate present in a biological sample of a depressed patient at a baseline is below a cut-off level, on the correlation the probability of occurrence of suicidal behavior within 12 months of the baseline was clearly confirmed.

Therefore, the assessment method for predicting and/or diagnosing suicidal behavior in depressed patients according to the present invention is classified according to whether the suicidal behavior is an increased suicidal severity or a fatal/non-fatal suicide attempt, and each method includes can be classified into a method of measuring one or more biomarkers for predicting suicidal behavior and a method of measuring all five biomarkers. That is, as the suicidal behavior with an increased suicidal severity, the first method of measuring one or more biomarkers for predicting suicidal behavior and the second method of measuring all five biomarkers can be classified, as the suicidal behavior with a fatal/non-fatal suicide attempt, a third method that measures one or more biomarkers for predicting suicidal behavior and a fourth method that measures all three biomarkers can be classified.

First, the first method includes a measurement step of measuring a concentration of a suicidal behavior prediction biomarker contained in a biological sample of a depressed patient at a baseline; and a decision step of determining a probability of an increased suicidal severity, depending on the concentration of the suicidal behavior prediction biomarker, and the second method includes a measurement step of measuring a concentration of each of five suicidal behavior prediction biomarkers contained in a biological sample of a depressed patient at a baseline, the five suicidal behavior prediction biomarkers including cortisol, interleukin-1 beta (IL-1β), homocysteine, total cholesterol, and folate; and a decision step of determining a probability of an increased suicidal severity, depending on the concentration of each of the suicidal behavior prediction biomarkers.

More specifically, in the first method, to measure suicidal behavior prediction biomarkers analyzed in the measurement stage, analyzing at least one biomarker selected from the group consisting of cortisol, interleukin-1beta, homocysteine, total cholesterol, and folic acid is sufficient, but the second method is different only in that all five biomarkers need to be analyzed in the measurement step, and the method of measuring the biomarker concentration in the measurement step in both method may be the same. That is, the analysis method used in the measurement step may include other known methods useful for identifying the presence of a biomarker for predicting and/or diagnosing suicidal behavior in depressed patients. In the present invention, the analysis method may be performed both in vitro and/or in vivo, but preferably the analysis method of the present invention is an in-vitro method based on a sample obtained from an individual and provided in vitro. As such, the biological sample may be selected from among a tissue and a body fluid including blood.

The decision step in both of the first and second method is performed by comparing the concentration of the suicidal behavior prediction biomarker measured in the measurement step with a preset cutoff level. But since the first method considers only one or more of the measured biomarker's concentration and the second method considers all five measured biomarker's concentrations, each method is described sequentially.

In the first method, the decision step may be determined that suicidal behavior, especially an increased suicidal severity is likely to occur when one or more of the measured concentration of the biomarker is lower than or higher than a preset cutoff level for each biomarker. That is, when the measured concentration of each of the cortisol, interleukin-1 beta (IL-1β), and homocysteine is higher than the preset cutoff level thereof, it is determined that an increased suicidal severity is likely to occur, and when the concentration of each of the total cholesterol and folate is lower than the preset cutoff level, it is determined that an increased suicidal severity is likely to occur.

In the decision step, as the number of the suicidal behavior prediction biomarkers indicating that an increased suicidal severity is likely to occur increases, the probability of an increased suicidal severity increases compared to a reference level. It was experimentally confirmed that when the number of the suicidal behavior prediction biomarkers indicating that an increased suicidal severity is likely to occur is one, the probability of an increased suicidal severity increases 2.1 times compared to the reference level, and when the number of the suicidal behavior prediction biomarkers is five, the probability of an increased suicidal severity increases 16.1 times compared to the reference level. Here, the cutoff level of each biomarker is determined through an experiment as described later and has a different value depending on the type of suicidal behavior prediction biomarker. That is, when the suicidal behavior prediction biomarker is the cortisol, the preset cutoff level is 11.7 μg/dL, when the suicidal behavior prediction biomarker is the IL-1β, the preset cutoff level is 0.99 μg/mL, when the suicidal behavior prediction biomarker is the homocysteine, the preset cutoff level is 11.1 μmol/dL, when the suicidal behavior prediction biomarker is the total cholesterol, the preset cutoff level is 155.0 mg/dL, and when the suicidal behavior prediction biomarker is the folate, the preset cutoff level is 6.05 ng/mL. The results obtained in the decision step of the first method show that the assessment method of this invention is sufficiently meaningful in that it can predict the possibility of suicidal behavior 2.1 times or more than the reference level even if only one biomarker is considered. However, considering all five biomarkers, it is shown that the probability of suicidal behavior improved by 16.1 times compared to the reference level can be predicted. Therefore, it can be seen that performing the second method can more accurately predict the possibility of suicidal behavior, especially an increased suicidal severity, in depression patients receiving drug therapy at the baseline and obtain significant results.

In the second method, the decision step needs to consider all five biomarkers, thus the decision step includes a reference point allocation step, a calculation step and a determination step. In the reference point allocation step, for each of the respective measured concentrations of the cortisol, IL-1β, and homocysteine, a reference point of 1 is allocated when the measured concentration is higher than the preset cutoff level thereof and a reference point of 0 is allocated when the measured concentration is lower than the preset cutoff level, and for each of the respective measured concentrations of the total cholesterol and folate, a reference point of 1 is allocated when the measured concentration is lower than the preset cutoff level and a reference point of 0 is allocated when the measured concentration is higher than the preset cutoff level. The calculation step calculates a total score of multiple successive biomarkers according to Formula 1 below.

total score of multiple successive biomarkers=1.108×A+0.700×B+0.331×C+0.193×D+0.282×E, [Formula 1]

wherein A is the reference point of the IL-1β, B is the reference point of the total cholesterol, C is the reference point of the cortisol, D is the reference point of the folate, and E is the reference point of the homocysteine. In the determination step, a probability of an increased suicidal severity is determined by finding a quartile in which the calculated total score is located.

Here, the preset cutoff level of each suicidal behavior prediction biomarker is the same as described above. Therefore, the measured concentrations of five suicidal behavior prediction biomarkers are compared with the cutoff level, and each reference score is given, and then a continuous multi-biomarker score can be calculated according to Equation 1. When the multi-biomarker score is within a range of 0 to 0.61, the score is located in the first quartile, when the multi-biomarker score is within a range of 0.62 to 1.31, the score is located in the second quartile, when the multi-biomarker score is within a range of 1.32 to 1.72, the score is located in the third quartile, and when the multi-biomarker score is within a range of 1.73 to 2.61, the score is located in the fourth quartile. As a result, the possibility of suicidal behavior, particularly an increased suicidal severity, can be more accurately predicted depending on where it is located in the first to fourth quartiles, i.e., the continuous multi-biomarker score.

In other words, it was confirmed that when the score is located in the first quintile, the probability of an increased suicidal severity is less than 6%, when the score is located in the second quartile, the probability of an increased suicidal severity increases 2.20 times compared to the first quartile, when the score is located in the third quartile, the probability of an increased suicidal severity increases 4.29 times compared to the first quartile, and when the score is located in the fourth quartile, the probability of an increased suicidal severity increases 6.20 times compared to the first quintile.

Next, the third method includes a measurement step of measuring the concentration of a suicidal behavior prediction biomarker contained in a biological sample of a depressed patient at a baseline; and a decision step of determining a probability of occurrence of a fatal/non-fatal suicide attempt according to the concentration of the suicidal behavior prediction biomarker. The fourth method includes a measurement step of measuring the concentration of each of three suicidal behavior prediction biomarkers contained in a biological sample of a depressed patient at a baseline, the three suicidal behavior prediction biomarkers including cortisol, total cholesterol, and folate; and a decision step of determining a probability of a fatal/non-fatal suicide attempt according to the measured concentration of each of the suicidal behavior prediction biomarkers.

More specifically, in the third method, to measure suicidal behavior prediction biomarkers analyzed in the measurement stage, analyzing at least one biomarker selected from the group consisting of cortisol, total cholesterol, and folic acid is sufficient, but the fourth method is different only in that all three biomarkers need to be analyzed in the measurement step, and the method of measuring the biomarker concentration in the measurement step in both method may be the same. That is, the analysis method used in the measurement step may include other known methods useful for identifying the presence of a biomarker for predicting and/or diagnosing suicidal behavior in depressed patients. In the present invention, the analysis method may be performed both in vitro and/or in vivo, but preferably the analysis method of the present invention is an in-vitro method based on a sample obtained from an individual and provided in vitro. As such, the biological sample may be selected from among a tissue and a body fluid including blood.

The decision step in both of the third and fourth method is performed by comparing the concentration of the suicidal behavior prediction biomarker measured in the measurement step with a preset cutoff level. But since the third method considers only one or more of the measured biomarker's concentration and the fourth method considers all three measured biomarker's concentrations, each method is described sequentially.

In the third method, the decision step may be determined that suicidal behavior, especially a fatal/non-fatal suicide attempt is likely to occur when one or more of the measured concentration of the biomarker is lower than or higher than a preset cutoff level for each biomarker. That is, when the measured concentration of the cortisol is higher than the preset cutoff level thereof, it is determined that a fatal/non-fatal suicide attempt is likely to occur, and when the concentration of each of the total cholesterol and folate is lower than the preset cutoff level, it is determined that a fatal/non-fatal suicide attempt is likely to occur.

In the decision step, as the number of the suicidal behavior prediction biomarkers indicating that a fatal/non-fatal suicide attempt is likely to occur increases, the probability of a fatal/non-fatal suicide attempt increases compared to a reference level. It was experimentally confirmed that when the number of the suicidal behavior prediction biomarkers indicating that a fatal/non-fatal suicide attempt is likely to occur is one, the probability of a fatal/non-fatal suicide attempt 3.3 times compared to the reference level, and when the number of the suicidal behavior prediction biomarkers is three, the probability of a fatal/non-fatal suicide attempt increases 63.3 times compared to the reference level. Here, the cutoff level of each biomarker is determined through an experiment as described later and has a different value depending on the type of suicidal behavior prediction biomarker. That is, when the suicidal behavior prediction biomarker is the cortisol, the preset cutoff level is 12.0 μg/dL, when the suicidal behavior prediction biomarker is the total cholesterol, the preset cutoff level is 154.0 mg/dL, and when the suicidal behavior prediction biomarker is the folate, the preset cutoff level is 5.95 ng/mL. The results obtained in the decision step of the third method show that the assessment method of this invention is sufficiently meaningful in that it can predict the possibility of suicidal behavior 3.3 times or more than the reference level even if only one biomarker is considered. However, considering all three biomarkers, it is shown that the probability of suicidal behavior improved by 63.3 times compared to the reference level can be predicted. Therefore, it can be seen that performing the fourth method can more accurately predict the possibility of suicidal behavior, especially a fatal/non-fatal suicide attempt, in depression patients receiving drug therapy at the baseline and obtain significant results.

In the fourth method, the decision step needs to consider all three biomarkers, thus the decision step includes a reference point allocation step, a calculation step and a determination step. In the reference point allocation step, for the measured concentration of the cortisol, a reference point of 1 is allocated when the measured concentration is higher than the preset cutoff level thereof and a reference point of 0 is allocated when the measured concentration is lower than the preset cutoff level thereof, and for each of the respective measured concentrations of the total cholesterol and folate, a reference point of 1 is allocated when the measured concentration is lower than the preset cutoff level thereof and a reference point of 0 is allocated when the measured concentration is higher than the preset cutoff level thereof. The calculation step calculates a total score of multiple successive biomarkers according to Formula 2 below.

total score of multiple successive biomarkers=1.215×B+1.843×C+1.010×D, [Formula 2]

wherein B is the reference point of the total cholesterol, C is the reference point of the cortisol, and D is the reference point of the folate. In the determination step, a probability of a fatal/non-fatal suicide attempt is determined by finding a quartile in which the calculated total score is located.

Here, the preset cutoff level of each suicidal behavior prediction biomarker is the same as described above. Therefore, the measured concentrations of three suicidal behavior prediction biomarkers are compared with the cutoff level, and each reference score is given, and then a continuous multi-biomarker score can be calculated according to Equation 2. When multi-biomarker score is 0, the score is located in the first quartile, when the multi-biomarker score is within a range of 0.01 to 1.21, the score is located in the second quartile, when the multi-biomarker score is within a range of 1.22 to 1.84, the score is located in the third quartile, and when the multi-biomarker score is within a range of 1.85 to 4.07, the score is located in the fourth quartile. As a result, the possibility of suicidal behavior, particularly a fatal/non-fatal suicide attempt, can be more accurately predicted depending on where it is located in the first to fourth quartiles, i.e., the continuous multi-biomarker score.

In other words, it was confirmed that when the score is located in the first quartile, the probability of a fatal/non-fatal suicide attempt is less than 0.5%, when the score is located in the second quartile, the probability of a fatal/non-fatal suicide attempt increases 0.99 times compared to the first quartile, when the score is located in the third quartile, the probability of a fatal/non-fatal suicide attempt increases 5.36 times compared to the first quartile, and when the score is located in the fourth quartile, the probability of a fatal/non-fatal suicide attempt increases 32.34 times compared to the first quartile.

In addition, the present invention pertains to a diagnostic kit for predicting suicidal behavior in depressed patients, which is used to determine a treatment strategy by predicting the probability that depressed patients will attempt suicidal behavior while receiving drug therapy at baseline. The diagnostic kit of the present invention includes a biomarker measurement unit configured to measure the concentration of one or more biomarkers contained in a biological sample of a depressed patient, the one or more biomarkers being selected from the group consisting of cortisol, interleukin-1 beta (IL-1β)), homocysteine, total cholesterol, and folate.

Here, the biomarker measurement unit measures the concentration of each of the biomarkers using a high-sensitivity bead panel utilizing antigen-antibody reactions, ELISA, ECLISA, or an enzymatic method, etc. In one embodiment, cortisol and folate are able to be measured using electrochemiluminescence Immunoassay, IL-1β is able to be measured using human high sensitivity T Cell magnetic bead panel or ELISA, homocysteine is able to be measured using chemiluminescent microparticle immunoassay, and total cholesterol is able to be measured using enzymatic methods using cholesterol oxidase.

In the diagnostic kit for predicting suicidal behavior in depressed patients of the present invention, when the suicidal behavior is an increased suicidal severity, it can be judged that the probability of an increased suicidal severity of patient whose concentration of one or more of cortisol, interleukin-1 beta (IL-1β), homocysteine are found to be above the cut-off level or a concentration of one or more of total cholesterol and folate below the cut-off level, is more than 2.1 times higher than the reference level, according to the first and second methods. Also, when the suicidal behavior is a fatal/non-fatal suicide attempt, the diagnostic kit of the present invention can be judged that the probability of a fatal/non-fatal suicide attempt of patient whose concentration of cortisol is found to be above the cut-off level or a concentration of one or more of total cholesterol and folate below the cut-off level, is more than 3.3 times higher than the reference level, according to the third and fourth methods.

The biological sample of the depressed patient used in the diagnostic kit for predicting suicidal behavior in depressed patients of the present invention may be serum. Also, the diagnostic kit may be implemented in a microarray.

### Example

1. Research Subject

This study was carried out as a component of the MAKE Biomarker discovery for Enhancing anTidepressant Treatment Effect and Response (MAKE BETTER) program. Details of the study have been published as a design paper (Kang et al., 2018) and the study was registered with cris.nih.go.kr (identifier: KCT0001332). All data on socio-demographic and clinical characteristics at baseline, and treatment related variables at follow-up examinations during the acute treatment phase (evaluated at 3, 6, 9, 12 weeks) and during the continuation treatment phases (evaluated at 6, 9, and 12 months) were obtained using a structured clinical report form (CRF) by clinical research coordinators who were blind to treatment modalities. These staff were trained in CRF implementation and data collection methods by the research psychiatrists. Patients' data were recorded on a CRF, registered on the website of the MAKE BETTER study within 3 days, and monitored by data management center personnel. This study was approved by the Chonnam National University Hospital Institutional Review Board (CNUH 2012-014).

The Eligibility Criteria of MARE BETTER Project

Inclusion criteria were: i) aged older than 7 years; ii) diagnosed with major depressive disorder (MDD), dysthymic disorder, or depressive disorder not otherwise specified (NOS), using the Mini-International Neuropsychiatric Interview (MINI) (Sheehan et al., 1998), a structured diagnostic psychiatric interview based on the Diagnostic and Statistical Manual of Mental Disorders, Fourth Edition (DSM-IV) criteria (APA, 1994); iii) Hamilton Depression Rating Scale (HAMD) (Hamilton, 1960) score 14; iv) able to complete questionnaires, understand the object of the study, and sign the informed consent form. Exclusion criteria were as follows: i) unstable or uncontrolled medical condition; ii) unable to complete the psychiatric assessment or comply with the medication regimen, due to a severe physical illness; iii) current or lifetime DSM-IV diagnosis of bipolar disorder, schizophrenia, schizoaffective disorder, schizophreniform disorder, psychotic disorder NOS, or other psychotic disorder; iv) history of organic psychosis, epilepsy, or seizure disorder; v) history of anticonvulsant treatment; vi) hospitalization for any psychiatric diagnosis apart from depressive disorder (e.g., alcohol/drug dependence); vii) electroconvulsive therapy received for the current depressive episode; viii) pregnant or breastfeeding.

2. Participants

Patients with depressive disorders were consecutively recruited from March 2012 to April 2017 from those who had visited the outpatient psychiatric department of Chonnam National University Hospital. All inclusion instances represented new treatment episodes i.e., taking newly initiated antidepressant treatment—whether depressive symptoms were first-onset or recurrent. Details of eligibility criteria are described in the online supplement. All participants reviewed the consent form and written informed consent was obtained. All patients gave written informed consent to participate in the study and use their data.

The study was conducted in accordance with the Helsinki Declaration of 1975, as revised in 2008 and approved by the Ethics Commission of the Chonnam National University Hospital Institutional Review Board (CNUH 2012-014) as it uses de-identified data.

3. Baseline Characteristics

(1) Serum Biomarkers

Participants were instructed to fast from the night before for morning blood sampling, and to sit for 25-45 min quietly and relax before blood samples were acquired. Serum samples were separated and immediately frozen at −80° C. at clinical laboratories of the CNUH. All laboratory measurements were conducted by the Global Clinical Central Lab (Yongin, Korea) blind to patients' status. Fourteen blood biomarkers representing six functional systems were selected based upon existing evidence (Lee & Kim 2011; Sudol & Mann 2017) and were measured using the following methods:

i) HPA Axis

cortisol: Cobas Cortisol II electrochemiluminescence Immunoassay (Roche, Vilvoorde, Belgium).

ii) Serotonergic

serotonin: ClinRep high-performance liquid chromatography kit (Recipe, Munich, Germany).

iii) Immune and Inflammatory

hsCRP: Tina-quant C-reactive protein (latex) high sensitive assay (Roche, Vilvoorde, Belgium).

TNF-α: QUANTIKINE® HS ELISA Human TNF-α Immunoassay (R&D Systems, Minneapolis, USA).

IL-1β, IL-6, IL-4, and IL-10: Human High Sensitivity T Cell Magnetic Bead Panel (EMD Millipore, Billerica, USA).

iv) Lipids

total cholesterol: L-type CHO M cholesterol oxidase method kit (Wako Pure Chemical Industries, Osaka, Japan).

leptin: Human Leptin ELISA (BioVendor Laboratory Medicine, Inc., Modrice, Czech Republic).

ghrelin: GHRELIN (Total) radioimmunoassay kit (EMD Millipore, Billerica, USA).

v) Nutritional

folate: Cobas Elecsys Folate III electrochemiluminescence Immunoassay (Roche, Vilvoorde, Belgium).

homocysteine: ARCHITECT Homocysteine 1L71 Kit (Abbot, Wiesbaden, Germany).

vi) Neuroplastic

BDNF: QUANTIKINE® ELISA Human BDNF Immunoassay (R&D Systems Inc., Minneapolis, USA).

(2) Suicidal Behaviors

Previous suicidal attempt was defined as the self-reported information of committing intentional self-harm before the baseline evaluation with at least some intention to die irrespective of the objective lethality (Posner et al. 2007). Equivocal intention to die at that time of an intentional self-harm act also defined as a suicide attempt. However, self-injurious behaviors with no suicidal intention or unknown intention were not included from the definition. Baseline suicidal severity was evaluated by the Brief Psychiatric Rating Scale (BPRS) (Overall & Gorham 1962) suicidality item score. Participants were asked “Have you felt that life wasn't worth living? Have you thought about harming or killing yourself? Have you felt tired of living or as though you would be better off dead? Have you ever felt like ending it all?” If participants reported suicidal ideation, further questions were asked “How often have you thought about this? Do you have a specific plan?”. Participants' self-report was recorded as a score 1-7, and divided into lower [score 1 (not present)˜3 (mild)] vs. higher [score 4 (moderate)˜7 (extremely severe)] suicidal severity groups.

(3) Covariates

Data on socio-demographic characteristics were obtained: age, sex, year of education, marital status (currently married or not), cohabitation status (living alone or not), religion (religious observance or not), occupational state (current employed or not), and monthly income degree (above or below 2,000 USD). Clinical characteristics assessed were composed of diagnoses of depressive disorders (MDD or other depressive disorders) with certain specifiers including melancholic and atypical features, onset age and illness duration, number of previous depressive episodes, duration of present episode, family history of depression, number of concurrent physical disorders (applying a questionnaire asking for 15 systems or diseases), and smoking status (current smoking or not). Assessment scales were administered for evaluating symptoms. Depressive and anxiety symptoms were evaluated by the Hospital Anxiety Depression Scale-depression subscale (HADS-D) and anxiety subscale (HADS-A) (Zigmond & Snaith, 1983), respectively; and screening for alcohol related problems by the Alcohol Use Disorders Identification Test (AUDIT) (Saunders et al. 1993). Higher scores indicate more severe symptomatology.

4. Stepwise Pharmacotherapy

Treatment steps and strategies have been previously published (Kim et al., 2020). Before the treatment commencement, a comprehensive examination was carried out for patients' clinical manifestations, illness severity, physical comorbidities and medication lists, and history of prior treatments. In the first step, patients received antidepressant medication, considering these patient data and existing treatment guidelines (Bauer et al., 2013; Malhi et al., 2015; Kennedy et al., 2016) for 3 weeks. General effectiveness and tolerability were evaluated for going ahead with next-step measurement-based treatments (Guo et al., 2015). In cases of inadequate improvement or intolerable adverse events, patients were directed to choose whether they would prefer to stay in the present step or get in the next step treatment with switching antidepressants (S), augmenting with other drugs (A), combination of other antidepressants (C), S+A, S+C, A+C, and S+A+C strategies. For settling treatment strategies, patient's opinion was given priority for maximising drug compliance and treatment outcomes (Swift & Callahan, 2009).

5. Prospective Suicidal Behaviors

For assessing “increased suicidal severity”, the BPRS suicidality item score was re-evaluated during the 12-month pharmacotherapy period at 3, 6, 9, and 12 weeks, and at 6, 9, and 12 months. Any instance of an increase in the score during the follow-up period compared to the baseline score was defined as increased suicidal severity. Fatal/non-fatal suicide attempt included suicidal attempt defined as above and death by suicide during the 12-month pharmacotherapy period.

6. Statistical Analysis

Baseline socio-demographic and clinical characteristics including assessment scales, and treatment step during the 12-month pharmacotherapy period were compared by presence of previous suicidal attempt and by lower vs. higher baseline suicidal severity groups using t-tests or λ² tests as appropriate. Covariates for the further adjusted analyses were selected from those characteristics associated at conventional levels of statistical significance (p<0.05) in these analyses and considering collinearity between the variables. For estimating the individual associations with prospective SBs, baseline serum biomarker levels were compared by the increased suicidal severity and by fatal/non-fatal suicide attempt during the 12-month pharmacotherapy using Mann-Whitney U tests. For those biomarkers shown significance (P<0.05), optimal cut-offs with sensitivities and specificities were calculated against the two SBs by using the area under receiver operating curve (AUROC) analysis. Odds ratios and 95% confidence intervals (ORs and 95% CIs) for the two SBs were estimated by the dichotomized optimal cut-offs of each biomarkers using logistic regression analysis after adjustment for relevant covariates.

Effects of multiple biomarkers on prospective SBs were evaluated in two ways. First, summed up scores were calculated from the significant biomarkers, and then associations between increased number of biomarkers and SBs were investigated using logistic regression analysis after adjustment for covariates. ORs and 95% CIs were calculated for each group with the 0 score as reference. Second, a multi-biomarker score was estimated using the following equation based on the significant biomarkers: H=(β1×biomarker A)+(β2×biomarkerB), and so on, where β1 and β2 denote the estimates of beta coefficients for biomarkers A and B, and were obtained by fitting the logistic regression model for each SB. Patients were categorized according to quartiles of the multi-biomarker score. ORs and 95% CIs were calculated for each group with the lowest quartile as reference. Tests for linear trends in ORs were carried out using the increased number of biomarkers and the increased quartiles of multi-biomarker score.

Additional analyses were carried out to investigate the values of biomarkers for discriminating previous and present SBs by using the same statistical models. All statistical tests were two-sided with a significance level of 0.05. Statistical analyses were carried out using the SPSS 21.0 and STATA 12.0 software.

7. Results

(1) Recruitment

The recruitment process is summarized in Figure Of 1262 participants evaluated at baseline, 1094 (86.7%) provided a blood sample for measuring serum biomarkers. Of these, 884 (80.8%) completed the 12-week acute treatment and were followed at least once from 6 to 12 months continuation treatment, and included the sample for prospective analyses. Descriptive characteristics of the baseline and followed up samples are summarized in supplementary Table S1. No significant differences in baseline characteristics were found between those with or without a blood sample. However, loss to follow-up at 12 months was significantly associated with unemployed status and melancholic features at baseline.

TABLE 1

| | Baseline sample | | Followed up sample | |
|---|---|---|---|---|
| | (N = 1094) | | (N = 884) | |
| Socio-demographic characteristics | | | | |
| Age, mean (SD)/range years | 56.9 (14.9)/17~85 | | 57.0 (14.8)/17~85 | |
| Gender, N (%) female | 753 | (68.8) | 607 | (68.7) |
| Education, mean (SD)/range years | 9.1 (4.8)/0~20 | | 9.1 (4.8)/0~20 | |
| Marital status, N (%) unmarried | 327 | (29.9) | 257 | (29.1) |
| Living alone, N (%) | 167 | (15.3) | 131 | (14.8) |
| Religious non-affiliation, N (%) | 483 | (44.1) | 388 | (43.9) |
| Unemployed status, N (%) | 316 | (28.9) | 242 | (27.4) |
| Monthly income, N (%) <2,000 USD | 653 | (59.7) | 522 | (59.0) |
| Clinical characteristics | | | | |
| Major depressive disorder, N (%) | 933 | (85.3) | 761 | (86.1) |
| Melancholic feature, N (%) | 165 | (15.1) | 141 | (16.0) |
| Atypical feature, N (%) | 69 | (6.3) | 55 | (6.2) |
| Age at onset, mean (SD)/range years | 50.9 (16.6)/8~85 | | 50.8 (16.4)/8~85 | |
| Duration of illness, mean (SD)/range years | 6.3 (9.3)/0~57 | | 6.4 (9.4)/0~57 | |
| Number of depressive episodes, mean (SD)/ | 2.3 (5.0)/0~40 | | 2.3 (5.0)/0~40 | |
| range | | | | |
| Duration of present episode, mean (SD)/range | 7.4 (10.4)/0~90 | | 7.6 (10.4)/0~90 | |
| months | | | | |
| Family history of depression, N (%) | 160 | (14.6) | 125 | (14.1) |
| Number of physical disorders, mean (SD)/ | 1.6 (1.3)/0~5 | | 1.7 (1.3)/0~5 | |
| range | | | | |
| Current smoker, N (%) | 45 | (4.1) | 37 | (4.2) |

| Assessment scales, mean (SD) | | | |
|---|---|---|---|
| Hospital Anxiety & Depression Scale- | 13.7 (3.9)/2~21 | 13.7 (3.9)/2~21 | |
| depression subscale | | | |
| Hospital Anxiety & Depression Scale- | 11.8 (4.0)/1~21 | 11.8 (4.0)/1~21 | |
| anxiety subscale | | | |
| Alcohol Use Disorders Identification Test | 5.4 (8.9)/1~46 | 5.4 (9.1)/1~46 | |
| Treatment step over 12-month, N (%) | | 303 | (37.3) |
| Step 1 | | 268 | (33.0) |
| Step 2 | | 157 | (19.3) |
| Step 3 | | 84 | (10.3) |

(2) Characteristics by Previous and Present Suicidal Behaviours

In the baseline sample (N=1094), previous suicide attempt and higher baseline suicidal severity were present in 96 (8.8%) and 362 (33.1%) participants, respectively. Characteristics are compared by these SBs in Table 2 and 3.

TABLE 2

| | Absent | | Present | | Statistical | |
|---|---|---|---|---|---|---|
| | (N = 998) | | (N = 96) | | coefficients | P-value |
| Socio-demographic characteristics | | | | | | |
| Age, mean (SD) years | 57.9 | (14.4) | 46.9 | (15.5) | t = +7.124 | <0.001 |
| Gender, N (%) male | 301 | (30.2) | 40 | (41.7) | X² = 5.404 | 0.020 |
| Education, mean (SD) years | 8.9 | (4.8) | 10.9 | (4.5) | t = −3.857 | <0.001 |
| Marital status, N (%) unmarried | 285 | (28.6) | 42 | (43.8) | X² = 9.646 | 0.002 |
| Living alone, N (%) | 150 | (15.0) | 17 | (17.7) | X² = 0.486 | 0.486 |
| Religious non-affiliation, N (%) | 422 | (42.3) | 61 | (63.5) | X² = 16.049 | <0.001 |
| Unemployed status, N (%) | 288 | (28.9) | 28 | (29.2) | X² = 0.004 | 0.949 |
| Monthly income, N (%) <2,000 USD | 609 | (61.0) | 44 | (45.8) | X² = 8.397 | 0.004 |
| Clinical characteristics | | | | | | |
| Major depressive disorder, N (%) | 843 | (84.5) | 90 | (93.8) | X² = 6.010 | 0.014 |
| Melancholic feature, N (%) | 156 | (15.6) | 9 | (9.4) | X² = 2.676 | 0.102 |
| Atypical feature, N (%) | 53 | (5.3) | 16 | (16.7) | X² = 19.112 | <0.001 |
| Age at onset, mean (SD) years | 53.2 | (16.1) | 38.0 | (16.5) | t = +8.810 | <0.001 |
| Duration of illness, mean (SD) years | 4.7 | (8.7) | 8.8 | (11.6) | t = −3.415 | 0.001 |
| Number of depressive episodes, mean | 1.0 | (1.4) | 2.1 | (2.0) | t = −5.426 | <0.001 |
| (SD) | | | | | | |
| Duration of present episode, mean (SD) | 7.2 | (10.4) | 9.1 | (9.7) | t = −1.682 | 0.093 |
| months | | | | | | |
| Family history of depression, N (%) | 146 | (14.6) | 14 | (14.6) | X² = 0.001 | 0.990 |
| Number of physical disorders, mean | 1.7 | (1.3) | 1.3 | (1.3) | t = +2.453 | 0.114 |
| (SD) | | | | | | |
| Current smoker, N (%) | 102 | (10.2) | 21 | (21.9) | X² = 15.037 | 0.001 |
| Assessment scales, mean (SD) scores | | | | | | |
| Hospital Anxiety & Depression Scale- | 13.6 | (3.9) | 14.3 | (4.2) | t = −1.777 | 0.076 |
| depression subscale | | | | | | |
| Hospital Anxiety & Depression Scale- | 11.7 | (4.0) | 12.8 | (4.3) | t = −2.492 | 0.013 |
| anxiety subscale | | | | | | |
| Alcohol Use Disorders Identification | 4.7 | (8.3) | 11.7 | (12.0) | t = −5.576 | <0.001 |
| Test | | | | | | |
| Treatment step over 12-month (N = | | | | | | |
| 884), N (%) | | | | | | |
| Step 1 | 303 | (37.3) | 23 | (31.9) | X² = 10.151 | 0.017 |
| Step 2 | 268 | (33.0) | 18 | (25.0) | | |
| Step 3 | 157 | (19.3) | 15 | (20.3) | | |
| Step 4 | 84 | (10.3) | 16 | (22.2) | | |

TABLE 3

| | 0~2 | | 3~7 | | Statistical | |
|---|---|---|---|---|---|---|
| | (N = 732) | | (N = 362) | | coefficients | P-value |
| Socio-demographic characteristics | | | | | | |
| Age, mean (SD) years | 58.1 | (14.0) | 54.6 | (16.2) | t = +3.508 | <0.001 |
| Gender, N (%) female | 503 | (68.7) | 250 | (69.1) | X² = 0.013 | 0.908 |
| Education, mean (SD) years | 9.1 | (4.7) | 9.0 | (4.9) | t = +0.287 | 0.774 |
| Marital status, N (%) unmarried | 198 | (27.0) | 129 | (35.6) | X² = 8.521 | 0.004 |
| Living alone, N (%) | 100 | (13.7) | 67 | (18.5) | X² = 4.399 | 0.036 |
| Religious non-affiliation, N (%) | 297 | (40.6) | 186 | (51.4) | X² = 11.473 | 0.001 |
| Unemployed status, N (%) | 195 | (26.6) | 121 | (33.4) | X² = 5.430 | 0.020 |
| Monthly income, N (%) <2,000 USD | 427 | (58.3) | 226 | (62.4) | X² = 1.690 | 0.194 |
| Clinical characteristics | | | | | | |
| Major depressive disorder, N (%) | 606 | (82.8) | 327 | (90.3) | X² = 10.985 | 0.001 |
| Melancholic feature, N (%) | 109 | (14.9) | 56 | (15.5) | X² = 0.063 | 0.801 |
| Atypical feature, N (%) | 38 | (5.2) | 31 | (8.6) | X² = 4.661 | 0.031 |
| Age at onset, mean (SD) years | 53.5 | (16.2) | 48.6 | (17.3) | t = +4.627 | <0.001 |
| Duration of illness, mean (SD) years | 4.6 | (8.6) | 6.0 | (9.9) | t = −2.341 | 0.020 |
| Number of depressive episodes, mean | 1.0 | (1.4) | 1.3 | (1.5) | t = −3.868 | <0.001 |
| (SD) | | | | | | |
| Duration of present episode, mean (SD) | 7.1 | (9.7) | 7.9 | (11.6) | t = −1.179 | 0.239 |
| months | | | | | | |
| Family history of depression, N (%) | 113 | (15.4) | 47 | (13.0) | X² = 1.168 | 0.280 |
| Number of physical disorders, mean (SD) | 1.6 | (1.2) | 1.7 | (1.3) | t = −0.522 | 0.602 |
| Current smoker, N (%) | 64 | (8.7) | 59 | (16.3) | X² = 8.914 | 0.003 |
| Assessment scales, mean (SD) scores | | | | | | |
| Hospital Anxiety & Depression Scale- | 12.9 | (4.0) | 15.2 | (3.4) | t = −9.961 | <0.001 |
| depression subscale | | | | | | |
| Hospital Anxiety & Depression Scale- | 10.9 | (3.9) | 13.6 | (3.7) | t = −11.123 | <0.001 |
| anxiety subscale | | | | | | |
| Alcohol Use Disorders Identification Test | 5.1 | (8.6) | 5.8 | (9.3) | t = −1.183 | 0.237 |
| Treatment step over 12-month (N = | | | | | | |
| 884), N (%) | | | | | | |
| Step 1 | 233 | (39.2) | 93 | (32.1) | X² = 7.271 | 0.007 |
| Step 2 | 193 | (32.5) | 93 | (32.1) | (linear) | |
| Step 3 | 110 | (18.5) | 62 | (21.4) | | |
| Step 4 | 58 | (9.8) | 42 | (14.5) | | |

Previous suicide attempt was significantly associated with younger age, male gender, higher education, unmarried status, no religion, higher monthly income, diagnosis of MDD, atypical depressive features, earlier age at onset, longer duration of illness, higher number of depressive episodes, current smoking status, higher scores on HADS-A and AUDIT, and higher treatment steps over 12-month. A higher baseline suicidal severity was significantly associated with younger age, unmarried status, living alone, no religion, unemployed status, diagnosis of MDD, atypical feature, earlier age at onset, longer duration of illness, higher number of depressive episodes, current smoking status, higher scores on HADS-D and HADS-A, and higher treatment steps over 12-month. Considering these associations and collinearity between the variables, covariates for further adjusted analyses were selected as follows: age, gender, living alone, religious affiliation, monthly income, atypical feature, number of depressive episodes, number of physical disorders, smoking status, scores on HADS-A and AUDIT, and treatment step.

For patients with an insufficient response or uncomfortable side effects, next treatment steps (1, 2, 3, and 4 or over) with alternative strategies (switching, augmentation, combination, and mixtures of these approaches) were administered considering measurements and patient preference at 3, 6, 9, and 12 weeks, and at 6, 9, and 12 months

(3) Individual Associations Between Serum Biomarkers and Prospective Suicidal Behaviors

Baseline levels of serum biomarkers were compared by increased suicidal severity during the 12-month pharmacotherapy in Table 4 and baseline levels of serum biomarkers were compared by fatal/non-fatal suicide attempt in Table 5.

TABLE 4

| | Increased suicidal severity^{a} | | | |
|---|---|---|---|---|
| Serum biomarkers | Absent(N = 729) | | Present(N = 155) | |
| Cortisol, μg/dL | 10.5 | (5.6) | 11.7 | (6.1)^{†} |
| Serotonin, ng/mL | 72.7 | (68.2) | 67.8 | (62.2) |
| High-sensitivity C-reactive | 0.5 | (1.0) | 0.5 | (1.0) |
| protein, mg/L | | | | |
| Tumor necrosis factor-α, | 0.6 | (0.4) | 0.6 | (0.4)* |
| pg/ml | | | | |
| Interleukin-1β, pg/mL | 1.1 | (0.7) | 1.1 | (0.6)^{‡} |
| Interleukin-6, pg/mL | 1.6 | (1.5) | 1.7 | (1.6) |
| Interleukin-4, pg/mL | 36.9 | (38.1) | 36.3 | (40.0) |
| Interleukin-10, pg/mL | 10.7 | (9.9) | 12.3 | (9.4)* |
| Total cholesterol, mg/dL | 178.0 | (49.0) | 167.0 | (59.0)* |
| Leptin, ng/mL | 5.7 | (6.1) | 5.3 | (6.4) |
| Ghrelin, pg/mL | 381.0 | (184.5) | 359.0 | (177.0) |
| Folate, ng/mL | 7.7 | (6.2) | 6.3 | (4.6)^{‡} |
| Homocysteine, μmol/L | 10.9 | (4.6) | 12.3 | (4.9)^{†} |
| Brain derived neurotrophic | 23.3 | (9.0) | 22.0 | (7.0)* |
| factor, ng/mL | | | | |

^{a}Increase in Brief Psychiatric Rating Scale suicidality item score during the follow-up compared to the baseline.
^{‡}P < 0.001;
^{†}P < 0.01;
*P < 0.05 by using Mann-Whitney U tests.

TABLE 5

| | Fatal/non-fatal suicide attempt | | | |
|---|---|---|---|---|
| Serum biomarkers | Absent(N = 846) | | Present(N = 38) | |
| Cortisol, μg/dL | 10.6 | (5.5) | 17.7 | (13.2)^{‡} |
| Serotonin, ng/mL | 72.1 | (66.6) | 64.5 | (108.4) |
| High-sensitivity C-reactive | 0.5 | (1.0) | 0.6 | (0.8) |
| protein, mg/L | | | | |
| Tumor necrosis factor-α, | 0.6 | (0.4) | 0.6 | (0.4) |
| pg/mL | | | | |
| Interleukin-1β, pg/mL | 1.1 | (0.7) | 0.9 | (1.1) |
| Interleukin-6, pg/mL | 1.6 | (1.5) | 1.7 | (1.3) |
| Interleukin-4, pg/mL | 37.0 | (38.7) | 35.1 | (35.2) |
| Interleukin-10, pg/mL | 10.8 | (9.8) | 12.1 | (12.0) |
| Total cholesterol, mg/dL | 177.0 | (51.0) | 152.0 | (54.8)* |
| Leptin, ng/mL | 5.7 | (6.1) | 5.0 | (4.6) |
| Ghrelin, pg/mL | 377.0 | (182.9) | 388.0 | (159.8) |
| Folate, ng/mL | 7.5 | (5.9) | 4.7 | (3.8)^{‡} |
| Homocysteine, μmol/L | 11.1 | (4.7) | 11.3 | (5.1) |
| Brain derived neurotrophic | 23.0 | (8.9) | 21.0 | (6.9) |
| factor, ng/mL | | | | |

^{a}Increase in Brief Psychiatric Rating Scale suicidality item score during the follow-up compared to the baseline.
^{‡}P < 0.001;
^{†}P < 0.01;
*P < 0.05 by using Mann-Whitney U tests.

As shown in Table 4, increased suicidal severity was significantly associated with higher levels of cortisol, TNF-α, IL-1β, IL-10, and homocysteine, but with lower levels of total cholesterol, folate, and BDNF. As shown in Table 5, fatal/non-fatal suicide attempt was significantly associated with higher cortisol level, but with lower levels of total cholesterol and folate.

For these biomarkers shown statistical significance, optimal cut-offs with sensitivities and specificities were obtained by AUROC analysis and were showed in Table 6 and 7 respectively.

TABLE 6

| | Increased suicidal severity^{a} | | | | |
|---|---|---|---|---|---|
| | Optimal | | | | |
| | cut-off | | OR (95% CI) | Sensitivity | Specificity |
| Cortisol | >11.7 | μg/dL | 1.49 (1.03-2.14) | 51.0% | 60.4% |
| Tumor necrosis | >0.57 | pg/mL | 1.38 (0.95-2.01) | 61.9% | 47.5% |
| factor-α | | | | | |
| Interleukin-1β | >0.99 | pg/mL | 2.73 (1.75-4.26) | 83.2% | 39.8% |
| Interleukin-10 | >9.53 | pg/mL | 1.50 (0.99-2.14) | 64.5% | 44.3% |
| Total cholesterol | <155.0 | mg/dL | 2.12 (1.45-3.11) | 75.2% | 43.2% |
| Folate | <6.05 | ng/mL | 1.49 (1.01-2.18) | 67.5% | 57.9% |
| Homocysteine | >11.1 | μmol/L | 1.60 (1.09-2.34) | 60.6% | 53.4% |
| Brain derived | <22.8 | ng/mL | 1.40 (0.97-2.01) | 52.7% | 58.1% |
| neurotrophic factor | | | | | |

^{a}Increase in Brief Psychiatric Rating Scale suicidality item score during the follow-up compared to the baseline.
Optimal cut-off values were obtained from the receiver operating characteristic curve.
Odds ratios (95% confidence intervals) [OR (95% CI)] were estimated by using logistic regression analyses after adjustment for age, gender, living alone, religious affiliation, monthly income, atypical feature, number of depressive episodes, number of physical disorders, smoking status, scores on Hospital Anxiety & Depression Scale-anxiety subscale and Alcohol Use Disorders Identification Test, and treatment step.

TABLE 7

| | Fatal/non-fatal suicide attempt | | | | |
|---|---|---|---|---|---|
| | Optimal | | | | |
| | cut-off | | OR (95% CI) | Sensitivity | Specificity |
| Cortisol | >12.0 | μg/dL | 7.56 (3.17-18.05) | 84.2% | 63.5% |
| Tumor necrosis | — | | | | |
| factor-α | | | | | |
| Interleukin-1β | — | | | | |
| Interleukin-10 | — | | — | — | — |
| Total cholesterol | <154.0 | mg/dL | 3.61 (1.74-7.46) | 74.6% | 57.9% |
| Folate | <5.95 | ng/mL | 2.67 (1.23-5.80) | 80.1% | 74.4% |

| | |
|---|---|
| Homocysteine | — |
| Brain derived | |
| neurotrophic factor | |

^{a}Increase in Brief Psychiatric Rating Scale suicidality item score during the follow-up compared to the baseline.
Optimal cut-off values were obtained from the receiver operating characteristic curve.
Odds ratios (95% confidence intervals) [OR (95% CI)] were estimated by using logistic regression analyses after adjustment for age, gender, living alone, religious affiliation, monthly income, atypical feature, number of depressive episodes, number of physical disorders, smoking status, scores on Hospital Anxiety & Depression Scale-anxiety subscale and Alcohol Use Disorders Identification Test, and treatment step.

In the logistic regression analysis after adjustment for age, gender, living alone, religious affiliation, monthly income, atypical feature, number of depressive episodes, number of physical disorders, smoking status, scores on HADS-A and AUDIT, and treatment step, increased suicidal severity was independently associated with above cut-offs of cortisol, IL-13, and homocysteine, and below cut-offs of total cholesterol and folate; and fatal/non-fatal suicide attempt was independently associated with above cut-off of cortisol, and below cut-offs of total cholesterol and folate.

(4) Multiple Biomarkers and Prospective Suicidal Behaviors

Incidences of two prospective SBs according to the increased number of biomarkers are described in the upper part of Table 8 and Table 9 respectively. As shown in Tables 8 and 9, the probability of SBs was increased incrementally with the increasing number of unfavourable biomarkers (all P-values for trend <0.001). Compared to the patients without any unfavourable biomarkers, the ORs (95% CIs) of those with all unfavourable biomarkers were 16.06 (2.87-90.03) and 63.29 (7.21-555.44) for increased suicidal severity and fatal/non-fatal suicide attempt, respectively in the same logistic regression model. Incidences of two prospective SBs according to the quartiles of multi-biomarker scores are described in the lower part of Table 8 and 9 respectively. The probability of SBs was increased incrementally with the higher quartile of multi-biomarker scores (all P-values for trend <0.001). The ORs (95% CIs) for the highest vs. lowest quartile of multi-biomarker scores were 6.20 (3.15-12.19) and 32.34 (4.20-248.99) for increased suicidal severity and fatal/non-fatal suicide attempt, respectively in the same logistic regression model.

TABLE 8

Increased suicidal severity^{a}

| | |
|---|---|
| | P-value |
| Present, | for |

| | | | |
|---|---|---|---|
| N | N (%) | OR (95% CI) | trend |

Number of serum
biomarkers

| 0 | 60 | 2 | (3.3) | Reference | | <0.001 |
|---|---|---|---|---|---|---|
| 1 | 214 | 14 | (6.5) | 2.10 | (0.46-9.55) | |
| 2 | 252 | 39 | (15.5) | 5.17 | (1.20-22.26) | |
| 3 | 236 | 59 | (25.0) | 9.08 | (2.12-38.85) | |
| 4 | 102 | 33 | (32.4) | 12.02 | (2.69-53.69) | |
| 5 | 20 | 8 | (40.0) | 16.06 | (2.87-90.03) | |
| Quartiles of multi- | | | | | | |
| biomarker scores | | | | | | |
| 1 (lowest) | 213 | 12 | (5.6) | Reference | | <0.001 |
| 2 | 219 | 27 | (12.3) | 2.20 | (1.07-4.51) | |
| 3 | 245 | 53 | (21.6) | 4.29 | (2.19-8.41) | |
| 4 | 207 | 63 | (30.4) | 6.20 | (3.15-12.19) | |

^{a}Increase in Brief Psychiatric Rating Scale suicidality item score during the follow-up compared to the baseline.
Odds ratios (95% confidence intervals) [OR (95% CI)] were estimated by using logistic regression analyses after adjustment for age, gender, living alone, religious affiliation, monthly income, atypical feature, number of depressive episodes, number of physical disorders, smoking status, scores on Hospital Anxiety & Depression Scale-anxiety subscale and Alcohol Use Disorders Identification Test, and treatment step.
^{b} For calculating the number of serum biomarkers, 0 (favorable) or 1 (unfavorable) score from the optimal cut-offs of each significant biomarker was generated, and then summed scores were estimated ranging from 0 to 5, with higher scores indicating more unfavorable condition.
^{c}For calculating the continuous multi-biomarker scores, the following equations were used: Increased suicidal severity = (1.108 × interleukin-1β) + (0.700 × total cholesterol) + (0.331 × cortisol) + (0.193 × folate) + (0.282 × homocysteine); and fatal/non-fatal suicide attempt = (1.215 × total cholesterol) + (1.843 × cortisol) + (1.010 × folate), respectively. Then, quartiles of the multi-biomarker scores were generated ranging from 1 to 4, with higher scores indicating higher risk.

TABLE 9

| | Fatal/non-fatal suicide attempt | | | |
|---|---|---|---|---|
| | | | | P-value |
| | | Present, | | for |
| | N | N (%) | OR (95% CI) | trend |
| Number of serum | | | | |
| biomarkers | | | | |
| 0 | 268 | 1 (0.4) | Reference | <0.001 |
| 1 | 381 | 5 (1.3) | 3.30 (0.38-28.89) | |
| 2 | 194 | 22 (11.3) | 29.24 (3.76-227.47) | |
| 3 | 41 | 10 (24.4) | 63.29 (7.21-555.44) | |
| 4 | | | | |
| 5 | | | | |
| Quartiles of multi- | | | | |
| biomarker scores | | | | |
| 1 (lowest) | 268 | 1 (0.4) | Reference | <0.001 |
| 2 | 137 | 0 (0.0) | 0.99 (0.00-0.00) | |
| 3 | 244 | 5 (2.0) | 5.36 (0.61-46.82) | |
| 4 | 235 | 32 (13.6) | 32.34 (4.20-248.99) | |

^{a}Increase in Brief Psychiatric Rating Scale suicidality item score during the follow-up compared to the baseline.
Odds ratios (95% confidence intervals) [OR (95% CI)] were estimated by using logistic regression analyses after adjustment for age, gender, living alone, religious affiliation, monthly income, atypical feature, number of depressive episodes, number of physical disorders, smoking status, scores on Hospital Anxiety & Depression Scale-anxiety subscale and Alcohol Use Disorders Identification Test, and treatment step.
^{b} For calculating the number of serum biomarkers, 0 (favorable) or 1 (unfavorable) score from the optimal cut-offs of each significant biomarker was generated, and then summed scores were estimated ranging from 0 to 5, with higher scores indicating more unfavorable condition.
^{c}For calculating the continuous multi-biomarker scores, the following equations were used: Increased suicidal severity = (1.108 × interleukin-1β) + (0.700 × total cholesterol) + (0.331 × cortisol) + (0.193 × folate) + (0.282 × homocysteine); and fatal/non-fatal suicide attempt = (1.215 × total cholesterol) + (1.843 × cortisol) + (1.010 × folate), respectively. Then, quartiles of the multi-biomarker scores were generated ranging from 1 to 4, with higher scores indicating higher risk.

(5) Serum Biomarkers for Previous and Present Suicidal Behaviors

Individual multiple associations of serum biomarkers with previous suicidal attempt and higher baseline suicidal severity were summarized in Table 10-12 using the same statistical models as for the prospective suicidal behaviors.

Previous suicidal attempt was independently associated with above cut-offs of hsCPR and TNF-α, and with below cut-offs of total cholesterol and folate; and higher baseline suicidal severity was independently associated with above cut-offs of hsCRP, IL-1β, and IL-4, and with the below cut-off folate after adjustment for age, gender, living alone, religious affiliation, monthly income, atypical feature, number of depressive episodes, number of physical disorders, smoking status, and scores on HADS-A and AUDIT (Table 11). Probabilities of these SBs were increased incrementally with the increasing number of unfavourable biomarkers and with the higher quartile of multi-biomarker scores (all P-values for trend <0.001) (Table 12).

TABLE 10

| | Previous suicidal attempt | | | | Higher baseline suicidal severity^{a} | | | |
|---|---|---|---|---|---|---|---|---|
| | Absent | | Present | | Absent | | Present | |
| Serum biomarkers | (N = 998) | | (N = 96) | | (N = 732) | | (N = 362) | |
| Cortisol, μg/dL | 10.6 | (5.7) | 11.1 | (6.6)* | 10.4 | (5.6) | 11.1 | (6.5)* |
| Serotonin, ng/mL | 73.8 | (66.1) | 66.9 | (84.5) | 74.5 | (64.6) | 67.4 | (77.9) |
| High-sensitivity C-reactive | 0.4 | (1.0) | 0.6 | (1.2)* | 0.4 | (1.0) | 0.6 | (1.2)^{‡} |
| protein, mg/L | | | | | | | | |
| Tumor necrosis factor-α, | 0.6 | (0.4) | 0.7 | (0.5)* | 0.6 | (0.4) | 0.6 | (0.5) |
| pg/mL | | | | | | | | |
| Interleukin-1β, pg/mL | 1.1 | (0.7) | 1.3 | (0.9)^{†} | 1.1 | (0.7) | 1.2 | (0.8)^{†} |
| Interleukin-6, pg/mL | 1.6 | (1.5) | 1.8 | (2.2) | 1.6 | (1.6) | 1.7 | (1.7) |
| Interleukin-4, pg/mL | 37.2 | (38.2) | 35.3 | (35.2) | 35.0 | (34.1) | 42.9 | (42.3)^{‡} |
| Interleukin-10, pg/mL | 10.7 | (9.7) | 11.0 | (10.8) | 10.3 | (9.9) | 11.0 | (9.6) |
| Total cholesterol, mg/dL | 178.0 | (51.3) | 168.0 | (63.5)* | 177.0 | (53.8) | 178.0 | (52.0) |
| Leptin, ng/mL | 5.8 | (6.2) | 5.6 | (5.7) | 6.0 | (6.2) | 6.0 | (6.2) |
| Ghrelin, pg/mL | 377.0 | (178.8) | 404.5 | (197.3) | 384.5 | (188.5) | 371.5 | (165.3) |
| Folate, ng/mL | 7.6 | (6.0) | 5.6 | (5.0)^{‡} | 7.8 | (6.1) | 6.8 | (5.5)^{‡} |
| Homocysteine, μmol/L | 10.9 | (4.8) | 10.9 | (3.8) | 11.0 | (4.7) | 10.8 | (4.7) |
| Brain derived neurotrophic | 23.5 | (8.6) | 22.0 | (8.4) | 23.7 | (8.8) | 22.7 | (8.5) |
| factor, ng/mL | | | | | | | | |

^{a}Brief Psychiatric Rating Scale suicidality item score 4 (moderate)~7 (extremely severe).
^{‡}P < 0.001;
^{†}P < 0.01;
*P < 0.05 by using Mann-Whitney U tests.

TABLE 11

| | Previous suicidal attempt | | | | | Higher baseline suicidal severity^{a} | | | | |
|---|---|---|---|---|---|---|---|---|---|---|
| | Optimal | | | | | Optimal | | | | |
| | cut-off | | OR (95% CI) | Sensitivity | Specificity | cut-off | | OR (95% CI) | Sensitivity | Specificity |
| Cortisol | >12.1 | μg/dL | 1.18 (0.74-1.88) | 47.9% | 64.4% | >10.1 | μg/dL | 1.18 (0.89-1.56) | 59.9% | 47.7% |
| High-sensitivity | >0.48 | mg/dL | 1.70 (1.06-2.72) | 60.4% | 53.7% | >0.60 | mg/dL | 1.85 (1.39-2.45) | 50.6% | 65.3% |
| C-reactive protein | | | | | | | | | | |
| Tumor necrosis | >0.55 | pg/mL | 1.98 (1.21-3.22) | 67.7% | 45.0% | — | | | | |
| factor-α | | | | | | | | | | |
| Interleukin-1β | >1.20 | pg/mL | 1.30 (0.81-2.08) | 59.4% | 59.7% | >0.93 | pg/mL | 1.43 (1.05-1.95) | 78.5% | 33.6% |
| Interleukin-4 | — | | | | | >28.7 | pg/mL | 1.53 (1.14-2.06) | 70.4% | 39.8% |
| Total | <144.0 | mg/dL | 2.64 (1.59-4.37) | 82.4% | 35.4% | — | | — | — | — |
| cholesterol | | | | | | | | | | |
| Folate | <6.15 | ng/mL | 1.89 (1.17-3.07) | 70.2% | 63.3% | <6.25 | ng/mL | 1.53 (1.15-2.05) | 65.4% | 59.4% |

^{a}Brief Psychiatric Rating Scale suicidality item score 4 (moderate)~7 (extremely severe).
Optimal cut-off values were obtained from the receiver operating characteristic curve.
Odds ratios (95% confidence intervals) [OR (95% CI)] were estimated by using logistic regression analyses after adjustment for age, gender, living alone, religious affiliation, monthly income, atypical feature, number of depressive episodes, number of physical disorders, smoking status, and scores on Hospital Anxiety & Depression Scale-anxiety subscale and Alcohol Use Disorders Identification Test.

TABLE 12

| Previous suicidal attempt | | | | Higher baseline suicidal severity^{a} | | | |
|---|---|---|---|---|---|---|---|
| | Present, | | P-value | | Present, | | P-value |
| N | N (%) | OR (95% CI) | for trend | N | N (%) | OR (95% CI) | for trend |

Number of serum
biomarkers

| | | | | | | | | | | |
|---|---|---|---|---|---|---|---|---|---|---|
| 0 | 191 | 5 | (2.6) | Reference | <0.001 | 74 | 14 | (18.9) | Reference | <0.001 |
| 1 | 328 | 21 | (6.4) | 2.48 (0.89-6.94) | | 240 | 56 | (23.3) | 1.19 (0.60-2.37) | |
| 2 | 326 | 27 | (8.3) | 2.90 (1.05-7.98) | | 372 | 114 | (30.6) | 1.86 (0.96-3.59) | |
| 3 | 214 | 34 | (15.9) | 7.09 (2.54-19.76) | | 299 | 116 | (38.8) | 2.35 (1.20-4.59) | |
| 4 | 35 | 9 | (25.7) | 13.83 (3.85-49.73) | | 109 | 62 | (56.9) | 4.84 (2.29-10.25) | |
| Quartiles of multi- | | | | | | | | | | |
| biomarker scores | | | | | | | | | | |
| 1 (lowest) | 276 | 10 | (3.6) | Reference | <0.001 | 295 | 66 | (22.4) | Reference | <0.001 |
| 2 | 208 | 12 | (5.8) | 1.73 (0.71-4.22) | | 275 | 80 | (29.1) | 1.43 (0.96-2.14) | |
| 3 | 346 | 26 | (7.5) | 1.99 (0.91-4.36) | | 188 | 62 | (33.0) | 1.75 (1.12-2.73) | |
| 4 | 264 | 48 | (18.2) | 5.84 (2.71-12.50) | | 336 | 154 | (45.8) | 2.74 (1.87-4.00) | |

^{a}Brief Psychiatric Rating Scale suicidality item score 4 (moderate)~7 (extremely severe).
Odds ratios (95% confidence intervals) [OR (95% CI)] were estimated by using logistic regression analyses after adjustment for age, gender, living alone, religious affiliation, monthly income, atypical feature, number of depressive episodes, number of physical disorders, smoking status, and scores on Hospital Anxiety & Depression Scale-anxiety subscale and Alcohol Use Disorders Identification Test.

8. Discussion

In this study of outpatients with depressive disorders, summations of the multiple serum biomarkers on cortisol, IL-1β, homocysteine, total cholesterol, and folate predicted increased suicidal severity, and on cortisol, total cholesterol, and folate predicted fatal/non-fatal suicide attempt during the 12-month pharmacotherapy, respectively, in a dose dependent manner. In addition, summations of hsCPR, TNF-α, total cholesterol, and folate, and of hsCRP, IL-1β, IL-4, and folate were significantly and incrementally associated with previous suicidal attempt and higher baseline suicidal severity, respectively. These associations were robust after adjustment for relevant covariates.

The role of individual blood biomarkers for explaining SBs has been unclear (Blasco-Fontecilla & Oquendo 2016), even in the meta-analytic results (Black & Miller, 2014; O'Connor et al., 2016; Wu et al. 2016; Gonzalez-Castro et al. 2021; Eisen et al. 2015). Similarly in this study, the individual biomarkers' sensitivities and specificities were unsatisfactory for clinical application, although their predictive and discriminant values for SBs estimated by ORs (95% CIs) were statistically significant (Table 6, Table 7 and Table 11). A particular observation of this study was that biomarkers in combination had significantly better and incremental predictive values for prospective SBs. The ORs of patients with all unfavourable biomarkers was 16.1 and 63.3 compared to those without, and those of the lowest vs. highest quartile of multi-biomarker scores were 6.2 and 32.3 for increased suicidal severity and fatal/non-fatal suicide attempt, respectively (Table 8 and 9). These were remarkable improvements for the ORs of individual markers shown 1.5-2.7 for increased suicidal severity and shown 2.7-7.6 for fatal/non-fatal suicide attempt. The similar context was shared with previous and present SBs (Table 12).

Many researchers have argued the importance and necessity of multiple biomarkers for explanation of SBs (Sudol & Mann 2017). The “omics” approaches might give a clue to incarnate a sensible solution, but have shown inconclusive mixed findings (Perroud et al. 2012; Le-Niculescu et al. 2013; Mullins et al. 2014). Some investigators have examined combinations of two biological risk factors simultaneously: a coupling of cortisol- and serotonin or cholesterol-related factors (Coryell and Schlesser, 2007; Jokinen et al., 2008; Jokinen et al., 2009; Mann et al., 2006). However, the explanatory power of the combinations for SBs was still unsatisfactory (Blasco-Fontecilla & Oquendo 2016). The role of multiple peripheral blood biomarkers has rarely been conducted and were not fully understood. An exception was that a variety of cytokines and chemokines were usually evaluated at once (Isung et al. 2012; Janelidze et al. 2013). However, these studies didn't evaluate the combined effects of the biomarkers. As far as we are aware, the present study was the first to investigate the multi-modal effects of blood biomarkers covering various functional systems on SBs.

Almost all previous studies in this kind have been conducted with cross-sectional case-control designs, comparing blood biomarker levels by histories of suicidal attempts or by severity of suicidality (O'Connor et al., 2016; Wu et al. 2016). Some studies have evaluated prospective increases in suicidal ideation during short term 8-12 week antidepressant treatment (Perroud et al. 2012). As far as we are aware, this was the first study investigated prospective associations of blood biomarker levels with fatal/non-fatal suicidal attempt as well as increased suicidal severity during the long-term 12-month treatment. Three serum biomarkers on cortisol, total cholesterol, and folate were identified as common significant predictors of the two prospective SBs. Cortisol has been extensively investigated as a SB biomarker for stress, but two recent meta-analyses reported overall no associations between cortisol levels and suicidal attempts due to the controversial findings among the studies (O'Connor et al., 2016; Hernandez-Diaz et al., 2020). Our finding of a lack of associations of cortisol levels with previous suicide attempts after adjustment was in agreement with these meta-analyses. From these findings, serum cortisol levels might be a predictive rather than a retrospective biomarker of SB in depressive patients receiving pharmacotherapy. Total cholesterol has also been investigated repeatedly based on the cholesterol-serotonin hypothesis. A meta-analysis identified an inverse association between serum total cholesterol levels and suicidality (Wu et al. 2016), consistent with our findings on the independent association with previous suicidal attempt. Folate is involved in methylation reactions necessary for the production of monoamine neurotransmitters, phospholipids, and nucleotides, Folate deficiency has been associated with depressive disorders (Kim et al. 2008) and folate intake has been associated with augmentation of antidepressant effects (Sarris et al. 2016). Folate thus might be associated with SB, while this issue has not been evaluated so far. The present findings suggested that serum total cholesterol and folate levels were significantly associated with prospective as well as previous SBs. However, these novel findings on prospective SBs need further replications.

Additionally, serum IL-1β and homocysteine levels were significantly associated with increased suicidal severity. A meta-analysis of cytokines and chemokines in suicidality reported that levels of IL-1β and IL-6 were significantly increased in blood samples of patients with suicidality compared with both patients without suicidality and healthy controls (Black & Miller, 2014), consistent with our findings on the baseline SB. In addition, our findings suggested that the serum IL-1β levels could also be used as a predictive marker of prospective SB. Homocysteine, also involved in methylation reactions as with folate, has been associated with depressive disorders (Kim et al. 2008), but has not been evaluated as a biomarker for suicide. Our significant findings on increased suicidal severity should be considered as empirical, since the statistical significance was found just one of five SBs and it was novel.

Previous suicidal attempt and higher baseline suicidal severity were independently associated hsCPR, TNF-α, total cholesterol, and folate, and hsCRP, IL-1β, IL-4, and folate, respectively. Other than biomarkers also predicting both two SBs (total cholesterol, and folate), all biomarkers associated with previous and present SBs were included in immune and inflammatory systems (hsCRP, IL-1β, and IL-4). As stated above, this kind of cytokine biomarkers were found to be significantly associated with previous and contemporary SBs in a meta-analysis (Black & Miller, 2014), probably due to effects of cytokines on kynurenine pathway of tryptophan degradation or on glutaminergic neurotransmission through tryptophan catabolism (Serafini et al. 2013). In addition, cytokine imbalance hypotheses were more widely accepted in depressive disorders (Dowlati et al. 2010), Since all the participants of this study were composed of patients with depressive disorders, it is liable that the cytokine markers might be overrepresented particularly for previous and contemporary SBs.

There are several limitations in this study. First, since the study design was naturalistic, treatment was decided by patient preference with a physician's guidance, rather than using a pre-established protocol. Thus, our results could just provide broad and general biomarkers for predicting SBs outcomes in the pharmacotherapy of depressive disorders. Second, biomarkers evaluated were frequently investigated and relatively well-known ones (Sudol & Mann, 2017) rather than novel ones, although the latter should be tested and replicated further. Third, the biomarkers were examined just at baseline despite of the fact that levels of some biomarkers were changed according to the pharmacological treatment responses (Martinotti et al. 2016; Yoshimura et al. 2009). However, the present study focused on predictive rather than reflective values of biomarkers for SBs. Fourth, there was a considerable sample attrition during the 12-month treatment period. Because of poor prognostic characteristics among participants who were lost to follow-up, such as unemployed status and melancholic features, these participants presumably would have attenuated (rather than exaggerated) the observed findings. Fifth, recruitment was carried out at a single site, which may limit the generalizability of the present findings, although a single centre study has potential strengths in terms of consistency in evaluation and treatment. Sixth, the number of fatal/non-fatal suicide during the 12-month pharmacotherapy was relatively small, and therefore more long-term follow-up is needed.

This study had several strengths, including its novel combined retrospective and prospective design for evaluation of suicidal behaviors. The sample size was large compared to previous biomarker studies, and participants were evaluated with a structured research protocol and well-recognized and standardized scales. As stated above, this study the first one to report on the predictive values of biomarkers in combination covering several functional systems. In addition, diverse covariates were considered, which could have influenced the study findings.

### 9. Conclusion

Identification of individuals at risk of suicide is mostly based upon subjective reports so far. The introduction of biomarkers would be beneficial by objective prediction of SBs. The present invention suggests that combinations of serum biomarkers on cortisol, total cholesterol, and folate, and plus IL-1β and homocysteine could considerably improve predictability of fatal/non-fatal suicide attempt and increased suicidal severity during the 12-month pharmacotherapy, respectively. These findings could be translated in clinical practice for treating outpatients with depressive disorders, since these were drawn from a naturalistic prospective design, maximising resemblance to real world clinical situations. Patients with these unfavourable biomarkers are recommended to be monitored frequently and treated carefully to prevent SBs. Given the multi-determined nature of suicide, a combination of blood-based, neuropsychological, and neuroimaging factors might yield a better estimate of suicide risk. The present novel findings with serum biomarker could be considered as bases for future comprehensive studies and prevention guidelines.

Consequently, Since the present invention can predict relatively accurately whether a depressed patient receiving drug therapy is likely to commit suicide in the future, it can not only contribute to a decision-making process with regard to patient-tailored effective treatment strategies, but also can be very helpful as a potential tool for treatment of patients with depression.

Although exemplary embodiments of the present invention have been disclosed for illustrative purposes, those skilled in the art will appreciate that various modifications and substitutions are possible without departing from the scope and spirit of the invention as disclosed in the accompanying claims.

What is claimed is:

1. A method for assessing a suicidal severity in a depressed patient, the method comprising:
measuring a concentration of a suicidal behavior prediction biomarker contained in a biological sample of the depressed patient, wherein the suicidal behavior prediction biomarker is one or more markers selected from the group consisting of cortisol, interleukin-1 beta (IL-1β), homocysteine, total cholesterol, and folate; and
determining a probability of an increased suicidal severity by comparing the measured concentration of the suicidal behavior prediction biomarker with a preset cutoff level thereof.

2. The method of claim 1, wherein the suicidal behavior prediction biomarker comprises the cortisol.

3. The method of claim 1, wherein the suicidal behavior prediction biomarker comprises the interleukin-1 beta (IL-1β).

4. The method of claim 1, wherein the suicidal behavior prediction biomarker comprises the homocysteine.

5. The method of claim 1, wherein the suicidal behavior prediction biomarker comprises the total cholesterol.

6. The method of claim 1, wherein the suicidal behavior prediction biomarker comprises the folate.

7. The method of claim 1, wherein the suicidal behavior prediction biomarker comprises the cortisol, the interleukin-1 beta (IL-1β), the homocysteine, the total cholesterol, and the folate; and
the determining comprises:
when the measured concentration of each of the cortisol, the interleukin-1 beta (IL-1β), and the homocysteine is higher than the preset cutoff level thereof, it is determined that the increased suicidal severity is likely to occur; and
when the concentration of each of the total cholesterol and the folate is lower than the preset cutoff level, it is determined that the increased suicidal severity is likely to occur.

8. The method of claim 7, wherein the preset cutoff level of the cortisol is 11.7 μg/dL;
the preset cutoff level of IL-1β is 0.99 pg/mL;
the preset cutoff level of the homocysteine is 11.1 μmol/dL;
the preset cutoff level of the total cholesterol is 155.0 mg/dL; and
the preset cutoff level of the folate is 6.05 ng/mL.

9. The method of claim 1, wherein the preset cutoff level of the cortisol is 11.7 μg/dL;
the preset cutoff level of IL-1β is 0.99 pg/mL;
the preset cutoff level of the homocysteine is 11.1 μmol/dL;
the preset cutoff level of the total cholesterol is 155.0 mg/dL; and
the preset cutoff level of the folate is 6.05 ng/mL.

10. The method of claim 7, wherein when the number of the suicidal behavior prediction biomarkers indicating that the increased suicidal severity is likely to occur is one, the probability of the increased suicidal severity increases 2.1 times compared to the reference level, and when the number of the suicidal behavior prediction biomarkers is five, the probability of the increased suicidal severity increases 16.1 times compared to the reference level.

11. The method of claim 7, wherein the determining comprises:
allocating a reference point by:
for each of the respective measured concentrations of the cortisol, the IL-1β, and the homocysteine, allocating a reference point of 1 each when the respective measured concentration is higher than the preset cutoff level thereof, and allocating a reference point of 0 each when respective the measured concentration is lower than the preset cutoff level thereof, and
for each of the respective measured concentrations of the total cholesterol and the folate, allocating a reference point of 1 each when the respective measured concentration is lower than the preset cutoff level thereof, and allocating a reference point of 0 each when the respective measured concentration is higher than the preset cutoff level thereof;
calculating a multi-biomarker score according to Formula 1 below:
multi-biomarker score=1.108×A+0.700×B+0.331×C+0.193×D+0.282×E [Formula 1]
wherein A is the reference point of the IL-1β, B is the reference point of the total cholesterol, C is the reference point of the cortisol, D is the reference point of the folate, and E is the reference point of the homocysteine; and
determining a probability of the increased suicidal severity by finding a quartile in which the calculated total score is located.

12. The method of claim 11, wherein the preset cutoff level of the cortisol is 11.7 μg/dL;
the preset cutoff level of the IL-1β is 0.99 pg/mL;
the preset cutoff level of the homocysteine is 11.1 μmol/dL;
the preset cutoff level of the total cholesterol is 155.0 mg/dL; and
the preset cutoff level of the folate is 6.05 ng/mL.

13. The method of claim 11, wherein when the multi-biomarker score is within a range of 0 to 0.61, the score is located in a first quartile,
when the multi-biomarker score is within a range of 0.62 to 1.31, the score is located in a second quartile,
when the multi-biomarker score is within a range of 1.32 to 1.72, the score is located in a third quartile, and
when the multi-biomarker score is within a range of 1.73 to 2.61, the score is located in a fourth quartile.

14. The method of claim 13, wherein when the score is located in the first quartile, the probability of the increased suicidal severity is less than 6%.

15. The method of claim 13, wherein when the score is located in the second quartile, the probability of the increased suicidal severity increases 2.20 times compared to the first quartile;
when the score is located in the third quartile, the probability of the increased suicidal severity increases 4.29 times compared to the first quartile; and
when the score is located in the fourth quartile, the probability of the increased suicidal severity increases 6.20 times compared to the first quartile.

16. A method for predicting suicidal behavior in a depressed patient, the method comprising:
measuring a concentration of a suicidal behavior prediction biomarker contained in a biological sample of the depressed patient, the suicidal behavior prediction biomarker comprising at least one selected from the group consisting of cortisol, total cholesterol, folate, and a combination thereof; and
determining a probability of occurrence of a fatal/non-fatal suicide attempt according to the concentration of the suicidal behavior prediction biomarker.

17. The method of claim 16, wherein the suicidal behavior prediction biomarker comprises the cortisol, the total cholesterol, and the folate.

18. The method of claim 17, wherein the determining comprises:
comparing the measured concentration of the suicidal behavior prediction biomarker with a preset cutoff level thereof,
wherein, when the concentration of the cortisol is higher than the preset cutoff level, it is determined that the fatal/non-fatal suicide attempt is likely to occur; and
when the concentration of each of the total cholesterol and the folate is lower than the preset cutoff level thereof, it is determined that the fatal/non-fatal suicide attempt is likely to occur.

19. The method of claim 18, wherein the preset cutoff level of the cortisol is 12.0 μg/dL;
the preset cutoff level of the total cholesterol is 154.0 mg/dL; and
the preset cutoff level of the folate is 5.95 ng/mL.

20. The method of claim 16, wherein the determining comprises determining that, as the number of suicidal behavior prediction biomarkers indicating that the fatal/non-fatal suicide attempt is likely to occur increases, the probability of occurrence of the fatal/non-fatal suicide attempt increases compared to a reference level.

21. The method of claim 20, wherein when the number of suicidal behavior prediction biomarkers indicating that the fatal/non-fatal suicide attempt is likely to occur is one, the probability of the fatal/non-fatal suicide attempt increases 3.3 times compared to the reference level, and
when the number of the suicidal behavior prediction biomarkers is three, the probability of the fatal/non-fatal suicide attempt increases 63.3 times compared to the reference level.

22. The method of claim 16, wherein the suicidal behavior prediction biomarker consists of the cortisol, the total cholesterol, and the folate.

23. The method of claim 22, wherein the determining comprises:
allocating a reference point by:
for the measured concentration of the cortisol, allocating a reference point of 1 when the measured concentration is higher than the preset cutoff level thereof, and allocating a reference point of 0 when the measured concentration is lower than the preset cutoff level thereof, and
for each of the respective measured concentrations of the total cholesterol and the folate, allocating a reference point of 1 each when the measured concentration is lower than the preset cutoff level thereof, and allocating a reference point of 0 when the measured concentration is higher than the preset cutoff level thereof;
calculating a multi-biomarker score according to Formula 2 below:
multi-biomarker score=1.215×B+1.843×C+1.010×D [Formula 2]
wherein B is the reference point of the total cholesterol, C is the reference point of the cortisol, and D is the reference point of the folate; and
determining a probability of the fatal/non-fatal suicide attempt by finding a quartile in which the calculated total score is located.

24. The method of claim 23, wherein the preset cutoff level of the cortisol is 12.0 μg/dL;
the preset cutoff level of the total cholesterol is 154.0 mg/dL; and
the preset cutoff level of the folate is 5.95 ng/mL.

25. The method of claim 23, wherein, when multi-biomarker score is 0, the score is located in a first quartile;
when multi-biomarker score is within a range of 0.01 to 1.21, the score is located in a second quartile;
when the multi-biomarker score is within a range of 1.22 to 1.84, the score is located in a third quartile; and
when the multi-biomarker score is within a range of 1.85 to 4.07, the score is located in a fourth quartile.

26. The method of claim 25, wherein, when the score is located in the first quartile, the probability of a fatal/non-fatal suicide attempt is less than 0.5%.

27. The method of claim 25, wherein, when the score is located in the second quartile, the probability of the fatal/non-fatal suicide attempt increases 0.99 times compared to the first quartile;
when the score is located in the third quartile, the probability of the fatal/non-fatal suicide attempt increases 5.36 times compared to the first quartile; and
when the score is located in the fourth quartile, the probability of the fatal/non-fatal suicide attempt increases 32.34 times compared to the first quartile.

28. A diagnostic kit for predicting suicidal behavior in a depressed patient, the diagnostic kit comprising:
a biomarker measurement unit configured to measure the concentration of one or more biomarkers contained in a biological sample of a depressed patient, the one or more biomarkers being selected from the group consisting of cortisol, interleukin-1 beta (IL-1β)), homocysteine, total cholesterol, and folate.

29. The kit of claim 28, wherein the biomarker measurement unit is configured to measure the concentration of each of the biomarkers using a high-sensitivity bead panel utilizing antigen-antibody reactions, ELISA, ECLISA, or an enzymatic method.

30. The kit of claim 28, wherein the diagnostic kit is a microarray.
